(19) United States
(12) Patent Application Publication
(10) Pub. No.: US 2026/0158165 A1
(43) Pub. Date: Jun. 11, 2026

(54) POLYPHENOL LIPID NANOPARTICLES AND METHODS OF USE

(71) Applicant: The University of North Carolina at Chapel Hill, Chapel Hill, NC (US)

(72) Inventors: Owen Fenton, Chapel Hill, NC (US); Yutian Ma, Chapel Hill, NC (US)

(21) Appl. No.: 19/128,368

(22) PCT Filed: Nov. 16, 2023
(86) PCT No.: PCT/US2023/079985

§ 371 Date: May 8, 2025

Related U.S. Application Data
(60) Provisional application No. 63/384,168, filed on Nov. 17, 2022.

(51) Int. Cl. A61K 48/00; A61K 47/69

(52) U.S. Cl. CPC A61K 48/0041; A61K 47/6915

(57) ABSTRACT

Disclosed herein are polyphenol lipid nanoparticles, including about 5-95 mol % ionizable lipid, about 5-30 mol % phospholipid, about 30-60 mol % sterol, about 1-5 mol % PEG lipid, and about 0.5-15 mol % polyphenol. Methods of making the lipid nanoparticles comprising a cargo, and methods of delivering a lipid nanoparticle comprising a cargo are provided. Methods of increasing endosomal escape of a lipid nanoparticle delivered to a cell are also provided and comprise inclusion of polyphenol in the lipid nanoparticle.

### STATEMENT OF PRIORITY

The present invention claims the benefit, under 35 U.S.C. § 119 (e), of U.S. Provisional Application No. 63/384,168, filed Nov. 17, 2022, the entire contents of which are incorporated by reference herein.

### FIELD OF INVENTION

The invention relates to polyphenol compounds, lipid nanoparticles comprising the polyphenol compounds and a cargo molecule, and methods of making them. The invention further relates to methods of using the novel polyphenol lipid nanoparticles to deliver cargo.

### BACKGROUND

In recent years, messenger RNAs (mRNAs) based therapeutics have provided a promising avenue for the treatment of various diseases, such as cancer, genetic disorders, and infection disease. One of the central goals of mRNA-based drugs is to develop drug delivery platforms for efficient delivery of mRNA into cells. Among the several delivery platforms used for mRNA delivery, lipid nanoparticles (LNPs) have emerged as a promising non-viral delivery technology for RNA based medicines as evidenced by the clinical successes of the COVID-19 mRNA vaccines developed by Moderna and Pfizer/BioNTech respectively. LNPs consist of a structural framework typically composed of four components: an ionizable lipid, a helper lipid, cholesterol, and a PEG lipid. Although it has evolved into a mature drug delivery platform, there is still significant room to improve current LNP technologies.

Even though lipid nanoparticles (LNPs) can deliver messenger RNA (mRNA) payloads into cells, their efficiency is often limited by endosomal trapping which prevents RNA payloads from acting therapeutically. Improving the percentage of RNA LNPs that can escape from endosomes and enter the cytoplasm is therefore an area of active research interest that could lead to improved safety profiles and reduced manufacturing costs of mRNA drugs.

### INVENTION SUMMARY

The present invention is based, in part, on the development of polyphenol lipid nanoparticles for the delivery of cargo, wherein the nanoparticles exhibit improved endosomal escape.

In one embodiment, a polyphenol lipid nanoparticle (LNP) is provided, comprising about 5-95 mol % ionizable lipid, about 5-30 mol % phospholipid, about 30-60 mol % sterol, about 1-5 mol % PEG lipid, and about 0.5-15 mol % polyphenol. In an aspect, the polyphenol is a flavonoid or a non-flavonoid.

The polyphenol may be selected from anthocyanins, dihydrochalcones, dihydroflavonols, flavanols, flavanones, flavonols, flavones, isoflavonoids, phenolic acids, xanthones, stilbenes, lignans, tannins, alkylmethoxyphenols, alkylphenols, curcuminoids, furanocoumarins, hydroxybenzaldehydes, hydroxybenzoketones, hydroxycinnamaldehydes, hydroxycoumarins, hydroxyphenylpropenes, methoxyphenols, naphtoquinones, phenolic terpenes, tyrosols, hydroxyphenolic acids, hydroxyphenylpentanoic acids, or any combination thereof. The polyphenol may be a tannin. In one preferred embodiment, the tannin is tannic acid.

In an embodiment, the ionizable lipid of the polyphenol LNP is selected from 9-heptadecanyl 8-{(2-hydroxyethyl)[6-oxo-6-(undecyloxy)hexyl]amino}octanoate (SM-102) and/or [(4-hydroxybutyl) azanediyl]di(hexane-6,1-diyl) bis(2-hexyldecanoate) (ALC-0315).

In an embodiment, the polyphenol LNP comprises a phospholipid selected from dioleoylphosphatidylethanolamine (DOPE), dioleoylphosphatidylcholine (DOPC), distearoylphosphatidylcholine (DSPC), or any combination thereof.

In an embodiment, the polyphenol LNP comprises a sterol that is cholesterol and/or phytosterol.

In an embodiment, the polyphenol LNP comprises a PEG lipid; in an aspect the PEG lipid is C14-PEG-2000.

The polyphenol LNP may comprise about 47 mol % SM-102, about 9 mol % DOPE, about 40 mol % cholesterol, about 2 mol % PEG lipid, and about 2 mol % tannic acid.

The polyphenol LNP may comprise about 44 mol % ALC-0315, about 9 mol % DOPE, about 40 mol % cholesterol, about 2 mol % PEG lipid and about 5 mol % tannic acid.

The polyphenol LNP may comprise a cargo. In an aspect, the cargo is a biologically active agent, imaging agent, or therapeutic agent. The cargo may be a nucleic acid, a protein, a complex of a nucleic acid and a protein, a carbohydrate, a lipid, or a small molecule, and salts thereof. In an aspect, the cargo nucleic acid is an RNA, for example, an mRNA, a guide RNA, antisense oligonucleotide, for example, comprising DNA or a DNA/RNA hybrid, or siRNA.

The cargo can be a gene modulating agent. In an aspect, the gene modulating agent is a gene editing system comprising a CRISPR-Cas system, a zinc finger nuclease, a TALEN, or a meganuclease.

Methods of delivering a cargo to a target cell are also provided and can comprise contacting the target cell with a polyphenol LNP described herein. The contacting may occur in vivo, ex vivo, or in vitro.

Methods of making a cargo-loaded nanoparticle are also provided, comprising: adding an organic phase comprising a polyphenol, a sterol, a PEG lipid, an ionizable lipid and a phospholipid to an aqueous phase comprising a cargo to make a mixture.

Methods of increasing endosomal escape of a LNP delivered to a cell are provided, comprising including a polyphenol in the LNP.

These and other aspects of the invention are set forth in more detail in the description of the invention below.

### BRIEF DESCRIPTION OF THE DRAWINGS

FIGS. 1A-1B. Schematic illustrations of (A) the formulation and delivery of tannic acid (TA) mRNA lipid nanoparticles [TA(+) mRNA LNPs] into cells. (B) In vivo delivery of TA(+) mRNA LNPs using intravenous (IV) and intramuscular (IM) routes of administration.

FIGS. 2A-2H. (A) Molar composition ratios for 1-TA(−) mRNA LNPs, 1-TA(+) mRNA LNPs, 2-TA(−) mRNA LNPs, and 2-TA(+) mRNA LNPs. (B) Chemical structures of representative molecular components contained within mRNA LNPs used in this study. (C) Size distribution by dynamic light scattering (DLS) and (D) mRNA encapsulation efficiency for 1-TA(−) mRNA LNPs, 1-TA(+) mRNA LNPs, 2-TA(−) mRNA LNPs, and 2-TA(+) mRNA LNPs. (E) Zeta potential measurements of 1-TA(−) mRNA LNPs, 1-TA(+) mRNA LNPs, 2-TA(−) mRNA LNPs, and 2-TA(+) mRNA LNPs at pH 4.0, 5.0, 6.0, 7.4. (F) Luciferase protein expression, (G) cell viability and (H) cell association in MDCK cells treated with 500 ng mL⁻¹ of 1-TA(−) mRNA LNPs, 1-TA(+) mRNA LNPs, 2-TA(−) mRNA LNPs, and 2-TA(+) mRNA LNPs per well after incubation for 24 h at 37° C. For cell viability study, ethanol was used as a “kill” control to confirm the functionality of XTT assay (where relevant, all data presented as mean±SD, n=3, ****p<0.0001, *p<0.05 and ns p>0.05 with 95% of confidence level from unpaired t-test).

FIGS. 3A-3K. (A) Schematic illustration of experimental plan to assess endosomal escape of fluorescently labeled LNPs. (B) Confocal laser scanning microscopy images of MDCK cells treated with ATTO-488-labeled versions of the 1-TA(−) mRNA LNPs, 1-TA(+) mRNA LNPs, 2-TA(−) mRNA LNPs, and 2-TA(+) mRNA LNPs for 24 h as well as their representative color scatter plots. Endo/lysosomes (red) were stained with LysoTracker Deep Red. Nuclei (blue) were stained with Hoechst 33342. Scale bars are 20 μm. Pearson Correlation Coefficient (PCC) analysis of (C) 1-TA(−) mRNA LNPs vs. 1-TA(+) mRNA LNPs and (D) 2-TA(−) mRNA LNPs vs. 2-TA(+) mRNA LNPs corresponding with FIG. 3B (data presented as the mean±SD, N>50, **P<0.01 and ****P<0.0001 with 95% of confidence level from unpaired t-test). (E) Schematic illustration of experimental plan to assess endosomal escape of non-fluorescent LNPs in cells whose endosomes contain the fluorescent dye calcein. Confocal laser scanning microscopy images of calcein containing MDCK cells incubated with (F) calcein (control), (G) calcein followed by 1-TA(−) mRNA LNPs, (H) calcein followed by 1-TA(+) mRNA LNPs, (I) calcein (control), (J) calcein followed by 2-TA(−) mRNA LNPs, and (K) calcein followed by 2-TA(+) mRNA LNPs for 24 h. Scale bars are 20 μm.

FIGS. 4A-4F. Representative luminescence biodistribution of (A) 1-TA(−) mRNA LNPs (n=3) and (B) 1-TA(+) mRNA LNPs via intravenous (IV) injection (n=3) and (C) 1-TA(−) mRNA LNPs (n=3) and (D) 1-TA(+) mRNA LNPs via intramuscular (IM) (n=3) with FLuc mRNA ex vivo. (E) Associated percent of bioluminescence of 1-TA(−) mRNA LNPs and 1-TA(+) mRNA LNPs via IV and IM injection across various organs including pancreas, spleen, liver, kidneys, uterus/ovaries, inguinal lymph node, lung, and heart. Data presented as mean±standard deviation (n=3). (F) Representative histology images of liver, spleen, and lung of mice after treatment with 1-TA(−) mRNA LNPs and 1-TA(+) mRNA LNPs via IV and IM injection (n=3). Scale bars are 50 μm.

FIGS. 5A-5D. Stability of (A) 1-TA(−) mRNA LNPs, (B) 1-TA(+) mRNA LNPs, (C) 2-TA(−) mRNA LNPs, and (D) 2-TA(+) mRNA LNPs in different 1× pH buffer media (4.0, 5.0, 6.0, 7.4) and DMEM+10% FBS, assessed by monitoring the diameter of LNPs for 192 h by DLS. The samples were incubated at 4° C. Data are shown as the mean±SD, n=3.

FIGS. 6A-6D. (A) Flow cytograms showing the cell association of control, 1-TA(−) mRNA LNPs, 1-TA(+) mRNA LNPs, 2-TA(−) mRNA LNPs and 2-TA(+) mRNA LNPs with MDCK cells. The untreated and treated cells were incubated at 37° C. for 24 h incubation. Histograms of MDCK cells treated with (6C) 1-TA(−) mRNA LNPs, 1-TA(+) mRNA LNPs and (6D) 2-TA(−) mRNA LNPs and 2-TA(+) mRNA LNPs at 488 green signals.

FIGS. 7A-7D. Size distribution of (A) 1-TA(−) mRNA LNPs, and (B) 1-TA(+) mRNA LNPs for intravenous injection; (C) 1-TA(−) mRNA LNPs, and (D) 1-TA(+) mRNA LNPs for intramuscular injection before and after dialysis, assessed by DLS.

FIG. 8. Size change of 1-TA(−) mRNA LNPs, and 1-TA(+) mRNA LNPs for intravenous injection and intramuscular injection before and after dialysis, respectively. Data are shown as the mean±SD, n=3.

FIGS. 9A-9B. Representative luminescence biodistribution of PBS via (A) intravenous (IV) and (B) intramuscular (IM) injection as negative control for FIGS. 4A-4D.

FIG. 10. Percent weight gain reported as mean±SD (n=3) 24 hours after respective intravenous and intramuscular dose into mice.

FIG. 11. Luciferase protein expression in MDCK cells treated with 500 ng mL⁻¹ of 1-TA(−) mRNA LNPs made with dioleoylphosphatidylethanolamine (DOPE), 1-TA(−) mRNA LNPs made with distearoylphosphatidylcholine (DSPC), 2-TA(−) mRNA LNPs made with DOPE, and 2-TA(−) mRNA LNPs made with DSPC after incubation for 24 h at 37° C. (All data presented as mean±SD, n=3, **p<0.01 with 95% of confidence level from unpaired t-test).

FIG. 12. Schematic illustration of the concept described in Example 2.

FIGS. 13A-13K. (A) Schematic illustration of mRNA encapsulated ILNPs formulation with different polyphenols via microfluidic chip. (B) Chemical structures of representative molecular excipients within mRNA LNPs used in this study. (C) Weight composition ratios for the formulation of mRNA LNPs with different polyphenols including GA, CAT, EGCG and TA with the same weight ratio. (D) Size/Diameter, (E) PDI, (F) zeta potentials and (G) FLuc mRNA encapsulation efficiency of LNPs with different polyphenols. (H) Size change of LNPs with different polyphenols in 1×pH 4, pH 7.4 DMEM+10% FBS and RPMI+10% FBS at 37° C. for 48 h. (I, J) Relationship and (K) heat-map format of dominant interactions to formulate polyphenol mediated LNPs, as analyzed from the size change of LNPs, GA LNPs, CAT LNPs, EGCG LNPs, and TA LNPs: hydrophobic interactions incubated in 500 mM and 100 mM Tween 20, hydrogen bonding incubated in 500 mM and 100 mM urea, and ionic interactions incubated in 500 mM and 100 mM NaCl. (All data presented as mean±SD, n=3).

FIGS. 14A-14H. Intracellular degradation and trafficking of LNPs with different polyphenols in B16-F10 and DC 2.4 cells. (A, C) Cellular association and (B, D) GMFI of B16-F10 cells treated with LNPs with different polyphenols at varying incubation times of 2, 4, 24 and 48 h. Representative confocal microscopy images showing the intracellular trafficking of LNPs with different polyphenols in (E) HepG2 and (F) DC 2.4 cells at varying incubation times of 2, 4, 24 and 48 h. Light gray, ATTO-488 labelled LNPs; medium gray, nuclei; dark gray, cell membrane. Scale bars are 10 μm. (G) Schematic illustration of the cell internalization mechanisms with corresponding related inhibitors used. (H) Study of the cell internalization mechanism of the LNPs with different polyphenols by monitoring the internalization efficiency in the presence of different endocytic inhibitors. Cells were treated with 500 ng mL⁻¹ of LNPs with different polyphenols at 37° C. (All data presented as mean±SD, n=3).

FIGS. 15A-15D. (A) Representative confocal images of DC 2.4 cells treated with ATTO-488 labelled LNPs with different polyphenols. Endo/lysosomes were stained with LysoTracker Deep Red. Nuclei were stained with Hoechst 33342. Scale bars are 10 μm. (B) Pearson Correlation Coefficient (PCC) analysis of ATTO-488 labelled LNPs with different polyphenols (Data presented as the mean±SD, n>50, ***p<0.001, *p<0.05 and ns p>0.05 with 95% of confidence level from unpaired t-test). (C) Titration curves of LNPs with different polyphenols in suspensions as a function of HCl. (D) Representative confocal images of DC 2.4 cells incubated with calcein, calcein and LNPs with different polyphenols in the absence (top row) and presence (bottom row) of inhibitor bafilomycin A₁ for 4 h at 37° C. LNPs with various polyphenols were not fluorescently labeled to avoid interference with the calcein signal. Scale bars are 10 μm. (All data presented as mean±SD, n=3).

FIGS. 16A-16D. In vitro FLuc expression of LNPs with different polyphenols treated with (A) B16-F10 and (B) DC 2.4 cells under 50 ng, 100 ng and 200 ng mRNA dose per well across desired time (2, 4, 24, 48 h). In vitro EPO expression of LNPs with different polyphenols treated with (C) B16-F10 and (D) DC 2.4 cells under 50 ng, 100 ng and 200 ng dose per well for 24 h. The concentration of EPO protein was measured via human EPO ELISA kit. Cells treated with naked EPO mRNA and fresh media were directly analyzed with the ELISA kits without further dilution. (All data presented as mean±SD, n=3).

FIGS. 17A-17F. (A) Representative luminescence biodistribution of LNPs with different polyphenols encapsulated with FLuc mRNA ex vivo (n=3) for each group. Mice injected with FLuc mRNA and PBS were used as positive and negative control, respectively. (B) Associated percent of bioluminescence and (C) total luminescence of FLuc mRNA encapsulated LNPs with different polyphenols across various organs including pancreas, spleen, liver, kidneys, uterus/ovaries, lung, and heart. (D) Human erythropoietin concentration after the injection of EPO mRNA encapsulated LNPs with different polyphenols for 24 h. Mice injected with EPO mRNA and PBS were used as positive and negative control, respectively. The concentration of human erythropoietin was characterized by Human EPO ELISA kits following the manufacturer's protocol. (E) Representative histology images of liver, spleen, and lung of mice after treatment with FLuc mRNA encapsulated LNPs via IV injection routes (n=3). Scale bars are 50 μm. (F) ALP, ALT, AST, BUN and CREAT blood testing results after the IV injection of FLuc mRNA encapsulated LNPs with different polyphenols (ns>0.05 with 95% of confidence level from unpaired t-test, and all data presented as mean±SD, n=3).

FIGS. 18A-18B. Cell viability of FLuc mRNA encapsulated LNPs, TA LNPs, GA LNPs, CAT LNPs, EGCG LNPs and TA LNPs with (A) B16-F10 and (B) DC 2.4 cells at 50 ng, 100 ng and 200 ng doses per well for desired times (2, 4, 24 and 48 h) at 37° C. 10× Triton dissolved in cell media was used as negative control. (Data are shown as the mean±SD, n=3).

FIGS. 19A-19B. Cell viability of EPO mRNA encapsulated LNPs, TA LNPs, GA LNPs, CAT LNPs, EGCG LNPs and TA LNPs with (A) B16-F10 and (B) DC 2.4 cells at 50 ng, 100 ng and 200 ng doses per well for 24 h at 37° C. Cells treated with naked EPO mRNA were used as positive control. 10× Triton dissolved in cell media was used as negative control. (Data are shown as the mean±SD, n=3).

FIGS. 20A-20I. (A) Molar composition ratios for the formulation of mRNA LNPs with different polyphenols including GA, CAT, EGCG and TA. To make the formulated LNPs with the same polyphenol molar ratio, the concentration of GA, CAT, EGCG and TA are 0.4, 0.7, 1.1 and 4 mg mL⁻¹, respectively. (B) Size/Diameter, (C) PDI, (D) zeta potentials and (E) FLuc mRNA encapsulation efficiency of LNPs with different polyphenols. In vitro FLuc expression of LNPs with different polyphenols formulated with same molar ratio (according to FIG. 19A) treated with (F) B16-F10 and (G) DC 2.4 cells under 50 ng, 100 ng and 200 ng mRNA dose per well over 24 h at 37° C. Cells treated with 50 ng, 100 ng and 200 ng naked mRNA were used as positive control. Cells treated with fresh media were used as negative control. (Data are shown as the mean±SD, n=3). Cell viability LNPs with different polyphenols formulated with same molar ratio (according to FIG. 20A) treated with (H) B16-F10 and (I) DC 2.4 cells under 50 ng, 100 ng and 200 ng mRNA dose per well over 24 h at 37° C. 1× Triton dissolved in cell media was used as negative control. (Data are shown as the mean±SD, n=3).

FIG. 21. Complete blood test result of LNPs, GA LNPs, CAT LNPs, EGCG LNPs, TA LNPs. Mice only injected with naked FLuc mRNA and PBS were used as positive and negative control. (Data are shown as the mean±SD, n=3).

FIG. 22. Complete blood test result of LNPs, GA LNPs, CAT LNPs, EGCG LNPs, TA LNPs. Mice only injected with naked FLuc mRNA and PBS were used as positive and negative control. (Data are shown as the mean±SD, n=3).

FIG. 23. Percent weight gain reported as mean±SD (n=3) 24 hours after respective intravenous dose of FLuc mRNA encapsulated LNPs into mice.

FIG. 24. Percent weight gain reported as mean±SD (n=3) 24 hours after respective intravenous dose of EPO mRNA encapsulated LNPs into mice.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc, and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, ±0.1%, or even 0.01% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., remodeling activity) of at least about 50% or more as compared to the activity of a polypeptide consisting of the recited sequence.

As used herein, the term “polypeptide” encompasses both peptides and proteins, unless indicated otherwise.

The terms “polynucleotide”, “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Non-limiting examples of optional substituents as referred to herein include halogen, alkyl, aralkyl, heteroalkyl, alkenyl, alkynyl, cycloalkyl, hydroxyl, alkoxy, amino, amido, nitro, cyano, amido, carbonyl, carboxyl, silyl, ether, alkylthio, sulfonyl, sulfonamido, ketone, aldehyde, ester, heterocyclyl, aryl, and heteroaryl.

As used herein, the term “alkyl”, used either alone or in compound words such as “haloalkyl” includes straight-chain or branched alkyl, such as methyl, ethyl, n-propyl, i-propyl, or the different butyl, pentyl or hexyl isomers, etc.

As used herein, “unsaturated” refers to compounds or structures having at least one degree of unsaturation (e.g., at least one double or triple bond).

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

All chiral, diastereomeric, racemic, and geometric isomeric forms of a structure are intended, unless specific stereochemistry or isomeric form is specifically indicated. All processes used to prepare compounds and intermediates made therein are encompassed by the present disclosure. All tautomers of shown or described compounds are also encompassed by the present disclosure.

When any variable (e.g., Ri) occurs more than one time in any constituent or formula for a compound, its definition at each occurrence is independent of its definition at every other occurrence. Thus, for example, if a group is shown to be substituted with one or more Ri moieties, then Ri at each occurrence is selected independently from the Markush group recited for Ri. Also, combinations of substituents and/or variables are permissible, but only if such combinations result in stable compounds within a designated atom's normal valency.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g., a human, non-human primate, lagomorph (e.g., rabbit), or rodent (e.g., mouse, rat). In some embodiments a human subject is a neonate, child, adult or geriatric subject.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Treat,” “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

The present invention relates to polyphenol lipid nanoparticles (LNPs), e.g., a polyphenol LNP comprising ionizable lipid, phospholipid, sterol, PEG lipid, and polyphenol. LNP formulations comprising an additional polyphenol component in addition to the traditional four component system are provided herein. In an embodiment, the polyphenol LNP may comprise about 5-95 mol % ionizable lipid, about 5-30 mol % phospholipid, about 30-60 mol % sterol, about 1-5 mol % PEG lipid, and about 0.5-15 mol % polyphenol. In some embodiments, the polyphenol LNP comprises about 46 mol % ionizable lipid, about 9 mol % phospholipid, about 39 mol % sterol, about 1 mol % PEG lipid, and about 4 mol % polyphenol, for example, tannic acid, gallic acid (GA), catechin (CAT), or Epigallocatechin gallate (EGCG).

In an embodiment, the polyphenol LNPs of the present invention are about 75 nm to about 1000 nm, about 90 nm to about 800 nm, about 95 nm to about 800 nm, about 100 nm to about 600 nm, about 100 nm to about 500 nm, about 100 nm to about 400 nm, about 100 nm to about 300 nm, about 100 nm to about 250 nm, or about 125 nm to about 225 nm average particle size.

The polyphenol LNPs of the present invention may have a polydispersity index (PDI) of between about 0 and 1, between about 0 and 0.8, between about 0 and 0.6, between about 0 and 0.5, between about 0 and 0.4, between about 0 and 0.3, between about 0 and 0.25, between about 0.05 and 0.35, between about 0.1 and 0.3, or between about 0.15 and 0.035.

The polyphenol LNPs may have a zeta potential of between −20 and +20 mV, between −10 and +10 mV, between −5 and +5 mV, between −2 and +2 mV, between −1 and +1 mV, between −2.0 and 1.5 mV, between −1.7 and 1.3 mV, between −1.5 and 1 mV, between −0.5 and +0.5 mV, or between −0.3 and +0.3 mV.

The polyphenol LNPs of the present invention can be provided with cargo for delivery to a cell, tissue or subject. Advantageously, the LNPs comprising polyphenols having a high affinity to interact with biomolecules such as proteins, polysaccharides, and nucleic acids, allowing for strong interaction between a cargo and the polyphenol LNP.

### Polyphenol

Polyphenols comprise multiple hydroxyl groups attached to aromatic carbons in a given molecule. In an aspect, the polyphenol comprises 1, 2, 3, 4, 5, 6, 7, 8, 9, 10 or more aromatic rings. In an aspect, the polyphenol comprises 10, 11, 12, 13, 14, 15, 16, 17, 18, 19, 20, 21, 22, 23, 24, 25 or more hydroxyl groups. Polyphenols constitute a class of naturally occurring compounds that are both cost-effective and diverse, encompassing more than 8000 unique molecules. In some embodiments, the polyphenol exhibits an affinity for interacting with biomacromolecules, including but not limited to proteins, polysaccharides, lipids, and nucleic acids, and may utilize both covalent and non-covalent bonding mechanisms.

In one embodiment, the polyphenol is naturally occurring. Polyphenols generally recognized as safe are generally preferred. In an aspect, the polyphenol comprises a sugar core. The polyphenol preferably has a solubility in an organic phase, e.g., ethanol phase, that allows for mixing of the nanoparticle components at a rate of at least 1 mol % and up to at least 15 mol %. In an embodiment, the polyphenol comprises a plurality of hydrogen bond donors and acceptors.

The polyphenol can be provided at about 0.5-15 mol %, or about 0.5%, about 1%, about 1.5%, about 2%, about 3%, about 4%, about 5%, about 6%, about 7%, about 8%, about 9%, about 10%, about 11%, about 12%, about 13%, about 14%, or about 15% in the polyphenol LNP or any range therein. In an aspect, the polyphenol can be provided at about 2-15 mol %, about 2-13 mol %, about 2-10 mol %, about 2-8 mol %, about 2-6 mol %, about 3-10 mol %, about 4-10 mol %, about 4-8 mol % or about 1-8 mol % in the polyphenol LNP. In an embodiment, the polyphenol LNP comprises one or more polyphenols, e.g., 1, 2, 3, 4, or 5 or more polyphenols. In an embodiment, the polyphenol is selected from anthocyanins, dihydrochalcones, dihydroflavonols, flavanols, flavanones, flavonols, flavones, isoflavonoids, phenolic acids, xanthones, stilbenes, lignans, tannins, alkylmethoxyphenols, alkylphenols, curcuminoids, furanocoumarins, hydroxybenzaldehydes, hydroxybenzoketones, hydroxycinnamaldehydes, hydroxycoumarins, hydroxyphenylpropenes, methoxyphenols, naphtoquinones, phenolic terpenes, tyrosols, hydroxyphenolic acids, hydroxyphenylpentanoic acids, or any combination thereof.

In an embodiment, the polyphenol is selected from malvidin 3-o-(6″-p-coumaroyl-glucoside), delphinidin 3-o-(6″-acetyl-galactoside), cyanidin 3-o-(6″-acetyl-galactoside), cyanidin 3-o-galactoside, cyanidin 3-o-glucoside, cyanidin 3-o-rutinoside, cyanidin 3-o-sophoroside, pelargonidin 3-o-glucoside, cyanidin 3-o-(6″-malonyl-glucoside), peonidin, peonidin 3-o-glucoside, peonidin 3-o-rutinoside, pelargonidin 3-o-rutinoside, pelargonidin, cyanidin, malvidin 3,5-o-diglucoside, cyanidin 3-o-glucosyl-rutinoside, pelargonidin 3-o-sophoroside, pelargonidin 3-o-glucosyl-rutinoside, cyanidin 3-o-(6″-succinyl-glucoside), pelargonidin 3-o-(6″-succinyl-glucoside), delphinidin 3-o-galactoside, delphinidin 3-o-glucoside, delphinidin 3-o-arabinoside, petunidin 3-o-galactoside, cyanidin 3-o-arabinoside, petunidin 3-o-glucoside, peonidin 3-o-galactoside, petunidin 3-o-arabinoside, malvidin 3-o-glucoside, malvidin 3-o-arabinoside, delphinidin 3-o-(6″-acetyl-glucoside), petunidin 3-o-(6″-acetyl-galactoside), peonidin 3-o-(6″-acetyl-galactoside), cyanidin 3-o-(6″-acetyl-glucoside), malvidin 3-o-(6″-acetyl-galactoside), petunidin 3-o-(6″-acetyl-glucoside), malvidin 3-o-(6″-acetyl-glucoside), peonidin 3-o-(6″-acetyl-glucoside), pelargonidin 3-o-arabinoside, delphinidin 3-o-rutinoside, cyanidin 3-o-sambubioside, pelargonidin 3-o-(6″-malonyl-glucoside), peonidin 3-o-(6″-p-coumaroyl-glucoside), cyanidin 3-o-xyloside, malvidin 3-o-galactoside, peonidin 3-o-arabinoside, petunidin 3-o-rutinoside, delphinidin 3-o-xyloside, petunidin 3-o-(6″-p-coumaroyl-glucoside), pelargonidin 3-o-galactoside, pelargonidin 3-o-sambubioside, delphinidin 3-o-sambubioside, cyanidin 3-o-xylosyl-rutinoside, vitisin a, delphinidin 3-o-(6″-p-coumaroyl-glucoside), pigment a, cyanidin 3-o-(6″-p-coumaroyl-glucoside), cyanidin 3-o-sambubioside 5-o-glucoside, cyanidin 3-o-(6″-caffeoyl-glucoside), cyanidin 3,5-o-diglucoside, pinotin a, delphinidin 3,5-o-diglucoside, pelargonidin 3,5-o-diglucoside, malvidin 3-o-(6″-caffeoyl-glucoside), cyanidin 3-o-(6″-dioxalyl-glucoside), delphinidin 3-o-glucosyl-glucoside, cyanidin 3-o-(6″-malonyl-3″-glucosyl-glucoside), delphinidin 3-o-feruloyl-glucoside, petunidin 3,5-o-diglucoside, petunidin 3-o-rhamnoside, cyanidin 3-o-diglucoside-5-o-glucoside, peonidin 3-o-diglucoside-5-o-glucoside, peonidin 3-o-(2-o-(6-o-(e)-caffeoyl-d-glucosyl)-d-glucoside)-5-o-d-glucoside, peonidin 3-o-sophoroside, peonidin 3-o-sambubioside, peonidin 3-o-sambubioside-5-o-glucoside, peonidin 3-o-xyloside, 4′-o-methylcyanidin 3-o-d-glucoside, cyanidin 3-o-(3″,6″-o-dimalonyl-glucoside), 4-o-methyldelphinidin 3-o-d-glucoside, isopeonidin 3-o-arabinoside, isopeonidin 3-o-galactoside, isopeonidin 3-o-glucoside, isopeonidin 3-o-rutinoside, isopeonidin 3-o-sambubioside, isopeonidin 3-o-xyloside, cyanidin 3-o-(2-o-(6-o-(e)-caffeoyl-d glucoside)-d-glucoside)-5-o-d-glucoside, 4′-o-methyldelphinidin 3-o-rutinoside, butein, xanthohumol, phloretin, phloridzin, phloretin 2′-o-xylosyl-glucoside, 3-hydroxyphloretin 2′-o-xylosyl-glucoside, 3-hydroxyphloretin 2′-o-glucoside, phloretin 2′-o-glucuronide, dihydroquercetin 3-o-rhamnoside, dihydroquercetin, dihydromyricetin 3-o-rhamnoside, (+)-catechin, (−)-epicatechin, (+)-gallocatechin, (−)-epigallocatechin, (−)-epicatechin 3-o-gallate, (−)-epigallocatechin 3-o-gallate, theaflavin, theaflavin 3-o-gallate, theaflavin 3′-o-gallate, theaflavin 3,3′-o-digallate, (+)-gallocatechin 3-o-gallate, (+)-catechin 3-o-gallate, procyanidin dimer b1, procyanidin dimer b2, procyanidin dimer b3, procyanidin dimer b4, procyanidin dimer b5, procyanidin dimer b7, prodelphinidin dimer b3, procyanidin trimer c1, procyanidin trimer eec, procyanidin trimer t2, procyanidin trimer c2, prodelphinidin trimer gc-gc-c, prodelphinidin trimer gc-c-c, prodelphinidin trimer c-gc-c, (−)-epicatechin-(2a-7) (4a-8)-epicatechin 3-o-galactoside, cinnamtannin a2, (+)-catechin 3-o-glucose, 3′-o-methylepicatechin, 4′-o-methyl-(−)-epicatechin 3′-o-glucuronide, epicatechin 3′-o-glucuronide, 3′-o-methylcatechin, 4′,4″-o-dimethylepigallocatechin 3-o-gallate, 4′-o-methylepigallocatechin, 4″-o-methylepigallocatechin 3-o-gallate, 4′-o-methylepicatechin, epigallocatechin 3-o-gallate-7-o-glucoside-4″-o-glucuronide, (−)-epigallocatechin 3-o-glucuronide, 3′-o-methyl-(−)-epicatechin 7-o-glucuronide, epicatechin 7-o-glucuronide, (−)-epigallocatechin 3′-o-glucuronide, (−)-epigallocatechin 7-o-glucuronide, 4′-o-methyl-(−)-epigallocatechin 3′-o-glucuronide, 4′-o-methyl-(−)-epigallocatechin 7-o-glucuronide, naringenin, eriodictyol, hesperetin, eriocitrin, hesperidin, naringin, narirutin, neoeriocitrin, neohesperidin, poncirin, didymin, narirutin 4′-o-glucoside, naringin 4′-o-glucoside, naringin 6′-malonate, isosakuranetin, naringenin 7-o-glucoside, pinocembrin, 8-prenylnaringenin, 6-prenylnaringenin, 6-geranylnaringenin, isoxanthohumol, eriodictyol 7-o-glucoside, sakuranetin, hesperetin 3′-o-glucuronide, hesperetin 7-o-glucuronide, hesperetin 3′-sulfate, homoeriodictyol, naringenin 4′-o-glucuronide, naringenin 5-o-glucuronide, naringenin 7-o-glucuronide, hesperetin 3′,7-o-diglucuronide, hesperetin 5,7-o-diglucuronide, apigenin, luteolin, diosmin, isorhoifolin, neodiosmin, rhoifolin, sinensetin, nobiletin, tangeretin, luteolin 7-o-diglucuronide, chrysin, luteolin 7-o-rutinoside, tetramethylscutellarein, luteolin 7-o-glucoside, apigenin 7-o-glucoside, apigenin 6,8-di-c-glucoside, apigenin 6,8-c-arabinoside-c-glucoside, apigenin 6,8-c-galactoside-c-arabinoside, luteolin 7-o-glucuronide, apigenin 7-o-glucuronide, luteolin 7-o-malonyl-glucoside, luteolin 6-c-glucoside, luteolin 7-o-(2-apiosyl-glucoside), luteolin 7-o-(2-apiosyl-6-malonyl)-glucoside, apigenin 7-o-apiosyl-glucoside, 7,3′,4′-trihydroxyflavone, 7,4′-dihydroxyflavone, geraldone, baicalein, apigenin 6-c-glucoside, hispidulin, cirsimaritin, 5,6-dihydroxy-7,8,3′,4′-tetramethoxyflavone, pebrellin, gardenin b, nepetin, jaceosidin, cirsilineol, eupatorin, 6-hydroxyluteolin, 6-hydroxyluteolin 7-o-rhamnoside, scutellarein, apigenin 7-o-(6″-malonyl-apiosyl-glucoside), chrysoeriol 7-o-apiosyl-glucoside, chrysoeriol 7-o-(6″-malonyl-apiosyl-glucoside), chrysoeriol 7-o-glucoside, chrysoeriol 7-o-(6″-malonyl-glucoside), apigenin 7-o-diglucuronide, rhoifolin 4′-o-glucoside, kaempferol, quercetin, quercetin 3-o-galactoside, quercetin 3-o-glucoside, quercetin 3-o-xyloside, quercetin 3-o-rhamnoside, quercetin 3-o-rutinoside, quercetin 3-o-sophoroside, quercetin 3-o-arabinoside, quercetin 3-o-xylosyl-glucuronide, isorhamnetin 3-o-glucoside 7-o-rhamnoside, isorhamnetin 3-o-rutinoside, kaempferol 3-o-glucuronide, isorhamnetin 7-o-rhamnoside, quercetin 3,4′-o-diglucoside, myricetin 3-o-rutinoside, myricetin, morin, kaempferide, myricetin 3-o-galactoside, myricetin 3-o-glucoside, quercetin 3-o-glucosyl-xyloside, quercetin 3-o-acetyl-rhamnoside, kaempferol 3-o-galactoside, galangin, isorhamnetin, kaempferol 3-o-glucoside, kaempferol 3-o-rutinoside, kaempferol 3-o-glucosyl-rhamnosyl-galactoside, kaempferol 3-o-glucosyl-rhamnosyl-glucoside, quercetin 3-o-glucosyl-rhamnosyl-galactoside, quercetin 3-o-glucosyl-rhamnosyl-glucoside, rhamnetin, isorhamnetin 3-o-glucoside, myricetin 3-o-rhamnoside, quercetin 3-o-rhamnosyl-galactoside, quercetin 3-o-glucuronide, isorhamnetin 3-o-glucuronide, myricetin 3-o-arabinoside, quercetin 7,4′-o-diglucoside, quercetin 4′-o-glucoside, isorhamnetin 4′-o-glucoside, 3,7-dimethylquercetin, kaempferol 3-o-sophoroside, kaempferol 3,7-o-diglucoside, kaempferol 3-o-sophoroside 7-o-glucoside, quercetin 3-o-(6″-malonyl-glucoside), kaempferol 3-o-(6″-malonyl-glucoside), kaempferol 3-o-rhamnoside, quercetin 3-o-(6″-malonyl-glucoside) 7-o-glucoside, patuletin 3-o-glucosyl-(1→6)-[apiosyl(1→2)]-glucoside, spinacetin 3-o-glucosyl-(1→6)-[apiosyl(1→2)]-glucoside, patuletin 3-o-(2″-feruloylglucosyl) (1→6)-[apiosyl(1→2)]-glucoside, spinacetin 3-o-(2″-p-coumaroylglucosyl) (1→6)-[apiosyl(1→2)]-glucoside, spinacetin 3-o-(2″-feruloylglucosyl) (1→6)-[apiosyl(1→2)]-glucoside, spinacetin 3-o-glucosyl-(1→6)-glucoside, jaceidin 4′-o-glucuronide, 5,3′,4′-trihydroxy-3-methoxy-6:7-methylenedioxyflavone 4′-o-glucuronide, 5,4′-dihydroxy-3,3′-dimethoxy-6:7-methylenedioxyflavone 4′-o-glucuronide, kaempferol 3-o-xylosyl-glucoside, kaempferol 3-o-acetyl-glucoside, quercetin 3-o-xylosyl-rutinoside, kaempferol 3-o-xylosyl-rutinoside, kaempferol 7-o-glucoside, kaempferol 3-o-galactoside 7-o-rhamnoside, kaempferol 3-o-(6″-acetyl-galactoside) 7-o-rhamnoside, quercetin 3-o-galactoside 7-o-rhamnoside, quercetin 3-o-(6″-acetyl-galactoside) 7-o-rhamnoside, kaempferol 3-o-(2″-rhamnosyl-galactoside) 7-o-rhamnoside, kaempferol 3-o-(2″-rhamnosyl-6″-acetyl-galactoside) 7-o-rhamnoside, 6,8-dihydroxykaempferol, isorhamnetin 3-o-galactoside, quercetin 3-o-rhamnosyl-rhamnosyl-glucoside, kaempferol 3-o-rhamnosyl-rhamnosyl-glucoside, methylgalangin, kaempferol 3,7,4′-o-triglucoside, 3-methoxynobiletin, 3-methoxysinensetin, quercetin 3′-o-glucuronide, quercetin 3′-sulfate, quercetin 4′-o-glucuronide, isorhamnetin 4′-o-glucuronide, daidzein, formononetin, genistein, biochanin a, glycitein, glycitin, 6″-o-acetyldaidzin, 6″-o-malonylgenistin, daidzin, genistin, 6″-o-acetylgenistin, 6″-o-acetylglycitin, 6″-o-malonyldaidzin, 6″-o-malonylglycitin, 2′,7-dihydroxy-4′,5′-dimethoxyisoflavone, 2-dehydro-o-desmethylangolensin, 2′-hydroxyformononetin, 3′,4′,7-trihydroxyisoflavan, 3′,4′,7-trihydroxyisoflavanone, 3′-hydroxydaidzein, 3′-hydroxy-o-desmethylangolensin, 4′,6,7-trihydroxyisoflavanone, 4′,7-dihydroxy-3′-methoxyisoflavan, 4′,7-dihydroxy-6-methoxyisoflavan, 4′-o-methylequol, 5,6,7,3′,4′-pentahydroxyisoflavone, 5,6,7,4′-tetrahydroxyisoflavone, 5,7,8,3′,4′-pentahydroxyisoflavone, 5,7,8,4′-tetrahydroxyisoflavone, 5′-hydroxy-o-desmethylangolensin, 5′-methoxy-o-desmethylangolensin, 6,7,3′,4′-tetrahydroxyisoflavone, 6,7,4′-trihydroxyisoflavone, 6′-hydroxyangolensin, 6′-hydroxy-o-desmethylangolensin, 7,8,3′,4′-tetrahydroxyisoflavone, 7,8,4′-trihydroxyisoflavone, angolensin, calycosin, daidzein 4′-o-glucuronide, daidzein 7-o-glucuronide, dihydrobiochanin a, dihydrodaidzein, dihydrodaidzein 7-o-glucuronide, dihydroformononetin, dihydrogenistein, dihydroglycitein, equol, formononetin 7-o-glucuronide, genistein 4′,7-o-diglucuronide, genistein 4′-o-glucuronide, genistein 5-o-glucuronide, genistein 7-o-glucuronide, glycitein 4′-o-glucuronide, glycitein 7-o-glucuronide, koparin, o-desmethylangolensin, orobol, prunetin, pseudobaptigenin, puerarin, daidzin 4′-o-glucuronide, irisolidone 7-o-glucuronide, tectorigenin 7-sulfate, tectorigenin 4′-sulfate, irisolidone, tectorigenin, tectoridin, 5,7-dihydroxy-8,4′-dimethoxyisoflavone, isotectorigenin, equol 7-o-glucuronide, equol 4′-o-glucuronide, 3′,4′,5,7-tetrahydroxyisoflavanone, 3′-o-methylequol, 6-o-methylequol, 3′-hydroxygenistein, 6-hydroxydihydrodaidzein, 3′-hydroxyequol, cis-4-hydroxyequol, 4′-methoxy-2′,3,7-trihydroxyisoflavanone, irilone, vestitone, sativanone, butin, 3′-hydroxymelanettin, melanettin, stevenin, violanone, isoliquiritigenin, dalbergin, 3′-o-methylviolanone, 8-hydroxydihydrodaidzein, secoisolariciresinol, matairesinol, lariciresinol, pinoresinol, syringaresinol, isolariciresinol, arctigenin, trachelogenin, medioresinol, 1-acetoxypinoresinol, sesamin, sesamolin, sesamolinol, sesaminol, sesamol, 7-hydroxymatairesinol, isohydroxymatairesinol, secoisolariciresinol-sesquilignan, cyclolariciresinol, 7-oxomatairesinol, todolactol a, conidendrin, 7-hydroxysecoisolariciresinol, nortrachelogenin, lariciresinol-sesquilignan, anhydro-secoisolariciresinol, dimethylmatairesinol, episesamin, episesaminol, enterodiol, enterolactone, sesaminol 2-o-triglucoside, schisandrin, gomisin d, schisandrol b, tigloylgomicin h, schisanhenol, schisantherin a, gomisin m2, deoxyschisandrin, schisandrin b, schisandrin c, 2-hydroxyenterodiol, 4-hydroxyenterodiol, 6-hydroxyenterodiol, 2-hydroxyenterolactone, 4-hydroxyenterolactone, 6-hydroxyenterolactone, 2′-hydroxyenterolactone, 4′-hydroxyenterolactone, 6′-hydroxyenterolactone, 5-hydroxyenterolactone, 7-hydroxyenterolactone, 4-ethylbenzoic acid, glycine, 1,3,5-trimethoxybenzene, vanilloylglycine, 4-vinylguaiacol, 4-ethylguaiacol, 4-vinylsyringol, 5-heneicosenylresorcinol, 5-heneicosylresorcinol, 5-heptadecylresorcinol, 5-nonadecenylresorcinol, 5-nonadecylresorcinol, 5-pentacosenylresorcinol, 5-pentacosylresorcinol, 5-pentadecylresorcinol, 5-tricosenylresorcinol, 5-tricosylresorcinol, 3-methylcatechol, 4-methylcatechol, 4-ethylcatechol, 4-vinylphenol, 4-ethylphenol, curcumin, demethoxycurcumin, bisdemethoxycurcumin, bergapten, psoralen, xanthotoxin, isopimpinellin, syringaldehyde, protocatechuic aldehyde, vanillin, 4-hydroxybenzaldehyde, gallic aldehyde, p-anisaldehyde, vanillin 4-sulfate, 3-methoxyacetophenone, 2,3-dihydroxy-1-guaiacylpropanone, paeonol, 2,4-dihydroxyacetophenone 5-sulfate, 2-hydroxy-4-methoxyacetophenone 5-sulfate, resacetophenone, norathyriol, ferulaldehyde, sinapaldehyde, coumarin, mellein, scopoletin, esculetin, esculin, umbelliferone, 4-hydroxycoumarin, urolithin a 3,8-o-diglucuronide, urolithin a, urolithin b, urolithin b 3-o-glucuronide, urolithin c, 2-methoxy-5-prop-1-enylphenol, anethole, eugenol, acetyl eugenol, [6]-gingerol, estragole, guaiacol, juglone, 1,4-naphtoquinone, carnosic acid, rosmanol, carnosol, epirosmanol, rosmadial, thymol, carvacrol, tyrosol, hydroxytyrosol, 3,4-dhpea-ac, p-hpea-ac, oleuropein, demethyloleuropein, 3,4-dhpea-ea, ligstroside, 3,4-dhpea-eda, hydroxytyrosol 4-o-glucoside, oleoside dimethylester, oleoside 11-methylester, p-hpea-eda, p-hpea-ea, oleuropein-aglycone, ligstroside-aglycone, tyrosol 4-sulfate, coumestrol, catechol, pyrogallol, phlorin, phenol, arbutin, 3,4-dihydroxyphenylglycol, lithospermic acid, salvianolic acid b, salvianolic acid c, salvianolic acid d, salvianolic acid g, isopropyl 3-(3,4-dihydroxyphenyl)-2-hydroxypropanoate, ellagic acid glucoside, protocatechuic acid, gallic acid, vanillic acid, gentisic acid, ellagic acid, 4-hydroxybenzoic acid, syringic acid, 5-o-galloylquinic acid, ellagic acid arabinoside, ellagic acid acetyl-xyloside, ellagic acid acetyl-arabinoside, benzoic acid, 2-hydroxybenzoic acid, 3-hydroxybenzoic acid, 2,3-dihydroxybenzoic acid, 2,4-dihydroxybenzoic acid, 4-hydroxybenzoic acid 4-o-glucoside, protocatechuic acid 4-o-glucoside, gallic acid 4-o-glucoside, 3,5-dihydroxybenzoic acid, 2,6-dihydroxybenzoic acid, gallic acid 3-o-gallate, gallic acid ethyl ester, valoneic acid dilactone, galloyl glucose, lambertianin c, sanguiin h-6, punicalagin, gallagic acid, 3-o-methylgallic acid, 4-o-methylgallic acid, 3,4-o-dimethylgallic acid, punicalin, 4-hydroxyhippuric acid, 3-hydroxyhippuric acid, 2-hydroxyhippuric acid, hippuric acid, paeoniflorin, vanillic acid 4-sulfate, p-coumaric acid, 5-p-coumaroylquinic acid, 4-p-coumaroylquinic acid, caffeic acid, feruloyl glucose, ferulic acid, caffeoyl tartaric acid, rosmarinic acid, o-coumaric acid, m-coumaric acid, sinapic acid, p-coumaroyl glucose, p-coumaroylquinic acid, 3-caffeoylquinic acid, verbascoside, 4-caffeoylquinic acid, p-coumaroyl tartaric acid, 2,5-di-s-glutathionyl caftaric acid, feruloyl tartaric acid, caffeic acid ethyl ester, cinnamoyl glucose, 5-caffeoylquinic acid, 3-p-coumaroylquinic acid, 2-s-glutathionyl caftaric acid, 5-feruloylquinic acid, 4-feruloylquinic acid, 3-feruloylquinic acid, 5-sinapoylquinic acid, 4-sinapoylquinic acid, 3-sinapoylquinic acid, 3,5-dicaffeoylquinic acid, isoferulic acid, caffeoyl glucose, p-coumaric acid 4-o-glucoside, caffeic acid 4-o-glucoside, ferulic acid 4-o-glucoside, p-coumaroyl tartaric acid glucosidic ester, p-coumaric acid ethyl ester, hydroxycaffeic acid, chicoric acid, 5-5′-dehydrodiferulic acid, 5-8′-dehydrodiferulic acid, 1,2-disinapoylgentiobiose, 1-sinapoyl-2-feruloylgentiobiose, 1,2-diferuloylgentiobiose, 1,2,2′-trisinapoylgentiobiose, 1,2′-disinapoyl-2-feruloylgentiobiose, 1-sinapoyl-2,2′-diferuloylgentiobiose, 1,2,2′-triferuloylgentiobiose, 8-o-4′-dehydrodiferulic acid, 5-8′-benzofuran dehydrodiferulic acid, 3,4-dicaffeoylquinic acid, 3,4-diferuloylquinic acid, 3,5-diferuloylquinic acid, 1,5-dicaffeoylquinic acid, 4,5-dicaffeoylquinic acid, avenanthramide 2p, avenanthramide 2c, avenanthramide 2f, p-coumaroyl malic acid, p-coumaroyl glycolic acid, cinnamic acid, caffeoyl aspartic acid, p-coumaroyl tyrosine, sinapine, avenanthramide k, 24-methylcholestanol ferulate, 24-methylcholesterol ferulate, 24-methyllathosterol ferulate, stigmastanol ferulate, sitosterol ferulate, schottenol ferulate, 24-methylenecholestanol ferulate, 3-o-methylrosmarinic acid, feruloylglycine, isoferulic acid 3-o-glucuronide, isoferulic acid 3-sulfate, ferulic acid 4-sulfate, ferulic acid 4-o-glucuronide, caffeic acid 4-sulfate, caffeic acid 3-sulfate, feruloyl c1-glucuronide, isoferuloyl c1-glucuronide, caffeic acid 3-o-glucuronide, caffeic acid 4-o-glucuronide, caffeoyl c1-glucuronide, 1,5-diferuloylquinic acid, 1-caffeoyl-5-feruloylquinic acid, 1-feruloyl-5-caffeoylquinic acid, 3,4-dihydroxyphenylacetic acid, 4-hydroxyphenylacetic acid, homovanillic acid, homoveratric acid, methoxyphenylacetic acid, 3-hydroxyphenylacetic acid, 2-hydroxyphenylacetic acid, phenacetylglycine, phenylacetic acid, 4-hydroxymandelic acid, 2-hydroxy-2-phenylacetic acid, homovanillic acid 4-sulfate, dihydro-p-coumaric acid, dihydrocaffeic acid, 3-hydroxy-3-(3-hydroxyphenyl) propionic acid, 3-(3,4-dihydroxyphenyl)-2-methoxypropionic acid, 3-hydroxyphenylpropionic acid, dihydroferulic acid 4-sulfate, dihydrocaffeic acid 3-o-glucuronide, dihydrocaffeic acid 3-sulfate, dihydroferulic acid, dihydroferulic acid 4-o-glucuronide, dihydrosinapic acid, dihydroferuloylglycine, danshensu, 3-methoxy-4-hydroxyphenyllactic acid, 3,4-dihydroxyphenyllactic acid methyl ester, hydroxydanshensu, 3-phenylpropionic acid, 3-hydroxy-4-methoxyphenyllactic acid, 4-hydroxyphenyl-2-propionic acid, 5-(3′-methoxy-4′-hydroxyphenyl)-γ-valerolactone, 4-hydroxy-(3′,4′-dihydroxyphenyl) valeric acid, 5-(3′,4′-dihydroxyphenyl)-valeric acid, 5-(3′,4′-dihydroxyphenyl)-γ-valerolactone, 5-(3′,4′,5′-trihydroxyphenyl)-γ-valerolactone, 5-(3′,5′-dihydroxyphenyl)-γ-valerolactone, 3-hydroxyphenylvaleric acid, 5-(3′,5′-dihydroxyphenyl)-γ-valerolactone 3-o-glucuronide, trans-resveratrol, piceatannol, e-viniferin, pterostilbene, d-viniferin, pallidol, piceatannol 3-o-glucoside, pinosylvin, resveratrol 5-o-glucoside, resveratrol, resveratrol 3-o-glucoside, 3,4,5,4′-tetramethoxystilbene, 3′-hydroxy-3,4,5,4′-tetramethoxystilbene, 4′-hydroxy-3,4,5-trimethoxystilbene, 4-hydroxy-3,5,4′-trimethoxystilbene, cis-resveratrol 3-o-glucuronide, cis-resveratrol 3-sulfate, cis-resveratrol 4′-o-glucuronide, cis-resveratrol 4′-sulfate, resveratrol 3-sulfate, trans-resveratrol 3,5-disulfate, trans-resveratrol 3,4′-disulfate, trans-resveratrol 3-o-glucuronide, trans-resveratrol 3-sulfate, trans-resveratrol 4′-o-glucuronide, trans-resveratrol 4′-sulfate, dihydroresveratrol, tannic acid, or any combination thereof. In some embodiments, the polyphenol is tannic acid, gallic acid (GA), catechin (CAT), or Epigallocatechin gallate (EGCG).

In an aspect, the polyphenol is of a discrete molecular weight. In some embodiments, the polyphenol is a polyphenol with a molecular weight greater than about 350 g/mol, for example, greater than about 375 g/mol, 400 g/mol, 425 g/mol, 450 g/mol, 475 g/mol, 500 g/mol, 525 g/mol, 550 g/mol, 575 g/mol, 600 g/mol, 625 g/mol, or 650 g/mol. In some embodiments, a higher molecular weight polyphenol, for example, greater than about 350 g/mol, such as tannic acid and EGCG, are utilized. In some embodiments, the higher molecular weight polyphenol provides improved in vivo performance of the delivery of mRNA or other cargo in polyphenol-containing LNPs. In some embodiments, the polyphenol is a polyphenol with a molecular weight lower than about 350 g/mol, for example, less than about 350 g/mol, 325 g/mol, 300 g/mol, 275 g/mol, 250 g/mol, 225 g/mol, or 200 g/mol. In some embodiments, a lower molecular weight polyphenol, for example, less than about 350 g/mol, such as gallic acid or catechin, is utilized. In some embodiments, the lower molecule weight polyphenol provides improved in vitro performance of the delivery of mRNA or other cargo in polyphenol-containing LNPs.

### Ionizable Lipid

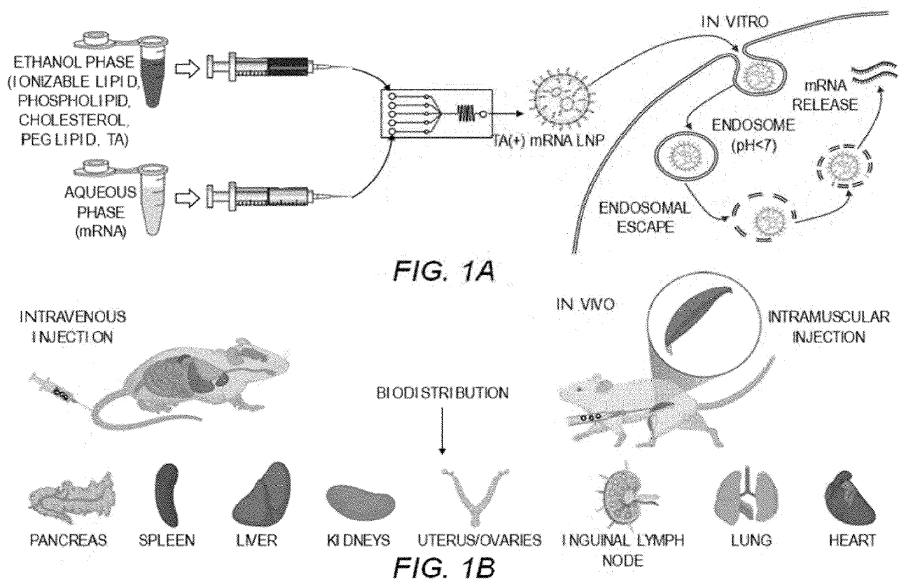

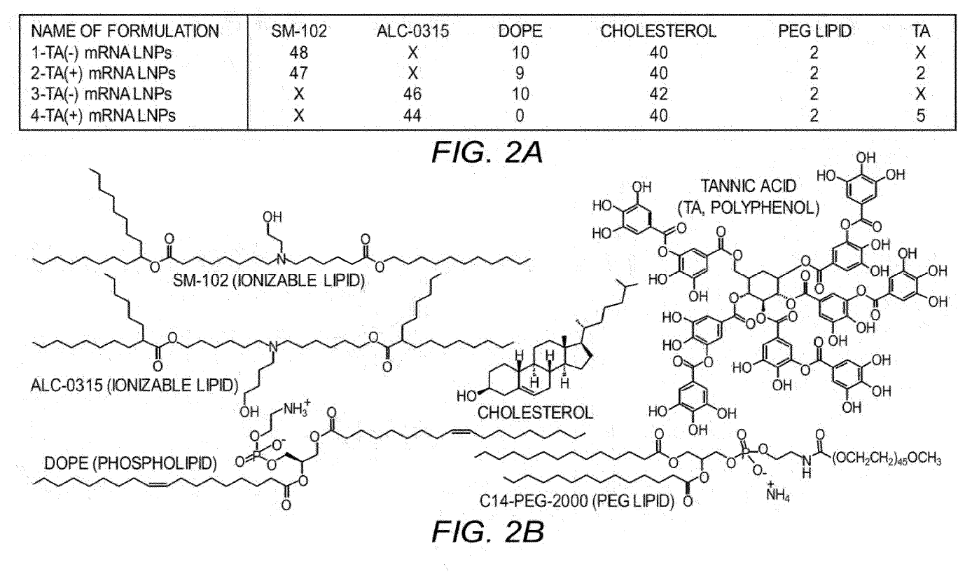

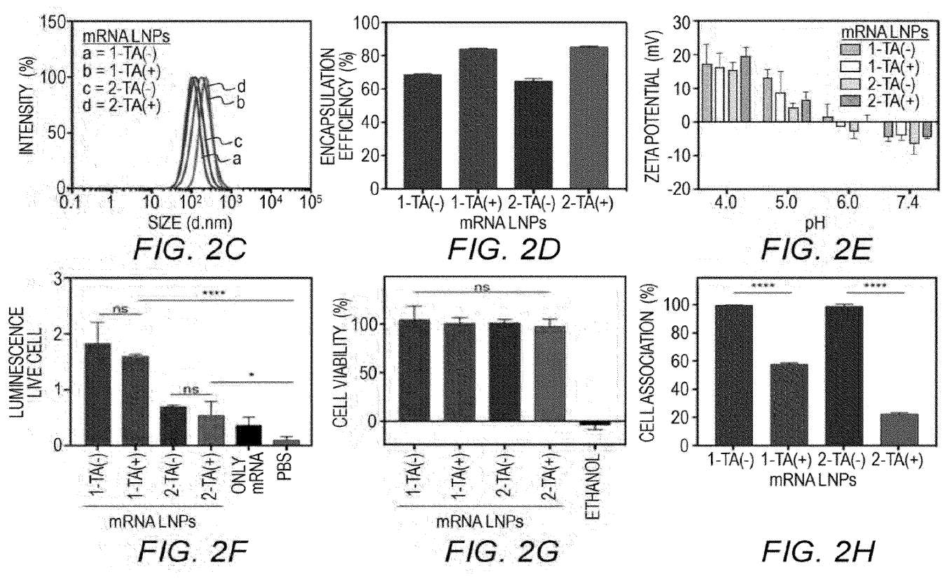

The ionizable lipid can be provided at about 5-95 mol %, e.g. about 5 mol %, about 10 mol %, about 15 mol %, about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, or about 50 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, about 60 mol %, about 61 mol %, about 62 mol %, about 63 mol %, about 64 mol %, about 65 mol %, about 66 mol %, about 67 mol %, about 68 mol %, about 69 mol %, about 70 mol %, about 71 mol %, about 72 mol %, about 73 mol %, about 74 mol %, about 75 mol %, about 76 mol %, about 77 mol %, about 78 mol %, about 79 mol %, about 80 mol %, about 81 mol %, about 82 mol %, about 83 mol %, about 84 mol %, about 85 mol %, about 86 mol %, about 87 mol %, about 88 mol %, about 89 mol %, about 90 mol %, about 91 mol %, about 92 mol %, about 93 mol %, about 94 mol %, or about 95 mol % or any range therein in the polyphenol LNP. The ionizable lipid can be provided at about 15-95 mol %, about 15-85 mol %, about 15-75 mol %, about 15-65 mol %, about 30-65 mol %, or about 35-50 mol % in the polyphenol LNP. In an aspect, the ionizable lipid can be provided at about 20-50 mol %, at about 25-50 mol %, at about 30-50 mol %, at about 30-45 mol % in the polyphenol LNP. In an embodiment, the polyphenol LNP comprises one or more ionizable lipids, e.g., 1, 2, 3, 4, or 5 or more ionizable lipids. In an aspect, the ionizable lipid is an unsaturated, multi-tail, polymeric, biodegradable, or branched-tail ionizable lipid. See, Han et al., Nature Communications volume 12, Article number: 7233 (2021), FIGS. 1 and 2, incorporated herein by reference. Example ionizable lipids include 1,2-dilinoleyl-N,N-dimethyl-3-aminopropane (DLinDMA), 2,2-dilinoleyl-4-dimethylaminoethyl-[1,3]-dioxolane (DLin-KC2-DMA), and dilinoleylmethyl-4-dimethylaminobutyrate (DLin-MC3-DMA). The ionizable lipid can preferably be selected from 9-heptadecanyl 8-{(2-hydroxyethyl)[6-oxo-6-(undecyloxy)hexyl]amino}octanoate (SM-102) and/or [(4-hydroxybutyl) azanediyl]di(hexane-6,1-diyl) bis(2-hexyldecanoate) (ALC-0315).

### Phospholipid

The phospholipid can be provided at about 5-30 mol %, about 8-30 mol %, about 8-25 mol %, about 8-20 mol %, or about 8-15 mol % in the polyphenol lipid nanoparticle. In an aspect, phospholipid can be provided at about 8-30 mol %, about 8-25 mol %, about 8-20 mol %, or about 8-15 mol % in the polyphenol lipid nanoparticle. The phospholipid can be provided at about 5%, about 6%, about 7%, about 8%, about 9%, about 10%, about 11%, about 12%, about 13%, about 14%, about 15% about 20 mol %, about 21 mol %, about 22 mol %, about 23 mol %, about 24 mol %, about 25 mol %, about 26 mol %, about 27 mol %, about 28 mol %, about 29 mol %, or about 30 mol % or any range therein in the polyphenol lipid nanoparticle. In an embodiment, the polyphenol LNP comprises one or more phospholipids, e.g., 1, 2, 3, 4, or 5 or more phospholipids. In an aspect, the phospholipid is dioleoylphosphatidylethanolamine (DOPE), dioleoylphosphatidylcholine (DOPC), distearoylphosphatidylcholine (DSPC), or any combination thereof.

### Sterol

The sterol can be provided at about 30-60 mol % in the polyphenol LNP. In an aspect the sterol can be provided at about 30 mol % to about 45 mol %, about 30 mol % to about 50 mol %, about 35 mol % to about 55 mol %, or about 35 mol % to about 45 mol % in the polyphenol LNP. The sterol can be provided at about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, about 55 mol %, about 56 mol %, about 57 mol %, about 58 mol %, about 59 mol %, or about 60 mol % or any range therein in the polyphenol LNP. In an embodiment, the polyphenol LNP comprises one or more sterols, e.g., 1, 2, 3, 4, or 5 or more sterols. In an aspect, the sterol is cholesterol and/or phytosterol.

### PEG Lipid

In an aspect, the PEG lipid is provided at about 1-5 mol % in the polyphenol lipid nanoparticle. In an aspect, the PEG lipid is provided at about 2 to about 5 mol %, about 2 to about 4 mol %, about 2 to about 3 mol %, about 1 to about 3 mol % in the polyphenol lipid nanoparticle. In an aspect, the PEG lipid is provided at about 1 mol %, about 2 mol %, about 3 mol %, about 4 mol %, or about 5 mol % or any range therein in the polyphenol lipid nanoparticle. In an embodiment, the polyphenol LNP comprises one or more PEG lipids, e.g., 1, 2, 3, 4, or 5 or more PEG lipids. The amount of PEG lipid can be tuned to adjust particle size, particle stability and aggregation, and zeta potential. Exemplary PEG lipids may comprise varying PEG lengths. In an aspect, the PEG is a 1,2-dimyristoyl-sn-glycero-3-phosphoethanolamine-N-[methoxy-(polyethylene glycol)] of varying lengths, for example, 1,2-dimyristoyl-sn-glycero-3-phosphoethanolamine-N-[methoxy-(polyethylene glycol)-350] (ammonium salt) (C14-PEG350), C14-PEG1000, C14-PEG2000, C18-PEG2000, C14-PEG3000, or any combination thereof. In an aspect, the PEG lipid is C14-PEG-2000, i.e., 1,2-dimyristoyl-sn-glycero-3-phosphoethanolamine-N-[methoxy-(polyethylene glycol)-2000] (ammonium salt).

### Cargo

The compositions disclosed herein may be provided with cargo molecules as nanoparticles for delivery. Agents including biologically active agents, imaging agents and therapeutic agents may be utilized as cargo molecules in the present invention. Example cargos may be a nucleic acid, a protein, a complex of a nucleic acid and a protein, a carbohydrate, a lipid, or a small molecule. The cargo may be formulated as a salt. One or two or more different cargos may be delivered by the delivery particles described herein. The cargo can be selected for the treatment of a disease or disorder, for the detection of a disease or disorder, or for monitoring of a disease or disorder. In an example embodiment, the cargo is an mRNA for delivery to a cell to increase expression of one or more proteins. In an embodiment, the cargo is an antisense oligonucleotide or siRNA for delivery to a cell to decrease expression or achieve gene silencing of one or more target sequences. The cargo may be an imaging agent or detectable label or may be a molecule comprising an imaging agent or detectable label, that can be delivered to a cell or tissue. In an aspect, the cargo is a genetic modulating agent such as a CRISPR-Cas system comprising a CRISPR-Cas protein, or a polynucleotide encoding the CRISPR-Cas protein, and a guide sequence specific for a target sequence that can be utilized for a variety of gene editing applications. One or both of the CRISPR-Cas system components can be provided in the same or in different particles. Accordingly, the particles comprising cargo will find use in therapeutic, detection, diagnostic, and other applications.

### Imaging Agents

Imaging agents, including reporter probes, can be delivered in the compositions, nanoparticles and methods of the present invention. The imaging agents and reporter probes may be associated with another molecule, covalently or otherwise, for delivery in the nanoparticle. In an aspect, a reporter probe may be associated with a nucleic acid specific for a target sequence in a cell, that can be delivered to a cell, tissue, or a subject in need thereof.

Imaging agents can include molecules suitable for any imaging modality, including positron emission tomography (PET) and single-photon emission computed tomography (SPECT), magnetic resonance imaging (MRI), ultrasound (US), and Computed Tomography (CT) imaging. Example agents can include magnetic agents including paramagnetic agents, such as gadolinium, and superparamagnetic iron-based agents. See, e.g., Xiao, et al. (2016). MRI contrast agents: Classification and application. Int'l J of Mol Med, 38, 1319-1326; doi: 10.3892/ijmm.2016.2744. Other contrast media may include iodinated, e.g., tri-iodinated benzene rings, or colloidal or micronized barium sulfate.

Reporter probes, including fluorescent, radioactive and other photo-emitting compounds, may also be delivered, including synthetic dye families such as tetramethylindo(di)-carbocyanines, fluorescein, and rhodamines. Biosensors may also be used, including in in vitro detection applications for bacterial, viral and other clinical applications. See, e.g., Castillo-Henriquez et al., Sensors (Basel). 2020 December; 20 (23): 6926; doi: 10.3390/s20236926.

### Biologically Active Agents

A biologically active agent, i.e., an agent that modulates an effect or activity in a cell, tissue, organ, or other biological media such as biological fluid, includes nucleic acids, proteins, small molecules, carbohydrates, lipids and complexes, salts thereof, and combinations thereof. The biologically active agent can be a genetic modifying agent. Representative nucleic acids include DNA, RNA, transposon DNA, antisense nucleic acids, ribozymes, plasmids, expression constructs, and RNA, such as mRNA, guide RNA, tRNA, ribosomal RNA, small nucleolar RNA, antisense oligonucleotides, or RNAi, such as siRNA, shRNA, and miRNA.

RNAi therapeutic agents comprise a polynucleotide that is complementary to a portion of the target sequence mRNA. In an example embodiment, the siRNA is a nucleic acid that can form a double stranded RNA with the ability to reduce or inhibit expression of a gene or target gene: each complementary sequence of the double stranded siRNA is about 15-50 nucleotides in length, and the double stranded siRNA is about 15-50 base pairs in length. A small hairpin RNA (shRNA) is also contemplated for use. The shRNA is an antisense strand of about 19 to about 25 nucleotides followed by a short nucleotide loop (approximately 5 to 9 nt) followed by the analogous sense strand. In an embodiment, an RNAi is a microRNA or miRNA, endogenous RNAs, some of which are known to regulate the expression of protein-coding genes at the posttranscriptional level. See, e.g., Lim et al Science 299, 1540 (2003), Lee and Ambros Science, 294, 862 (2001), Lau et al., Science 294, 858-861 (2001), Lagos-Quintana et al, RNA, 9, 175-179 (2003).

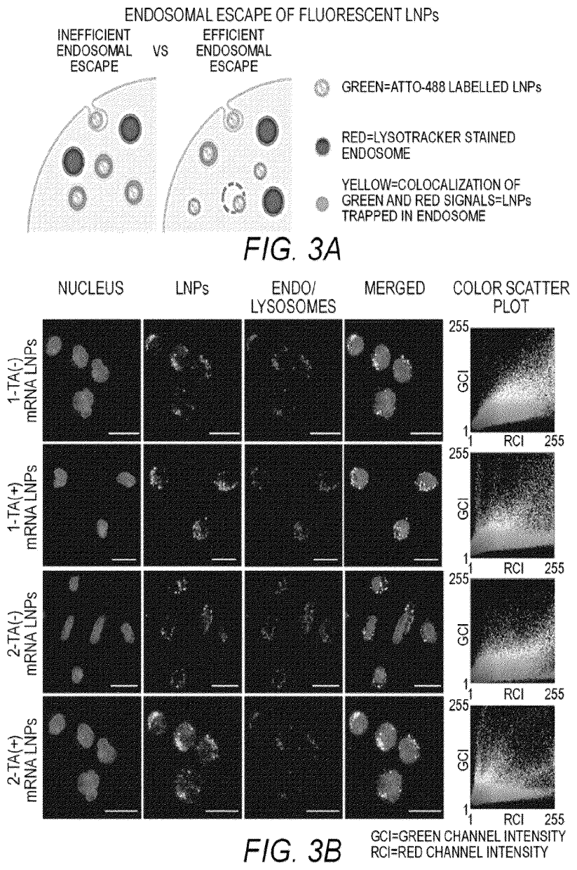

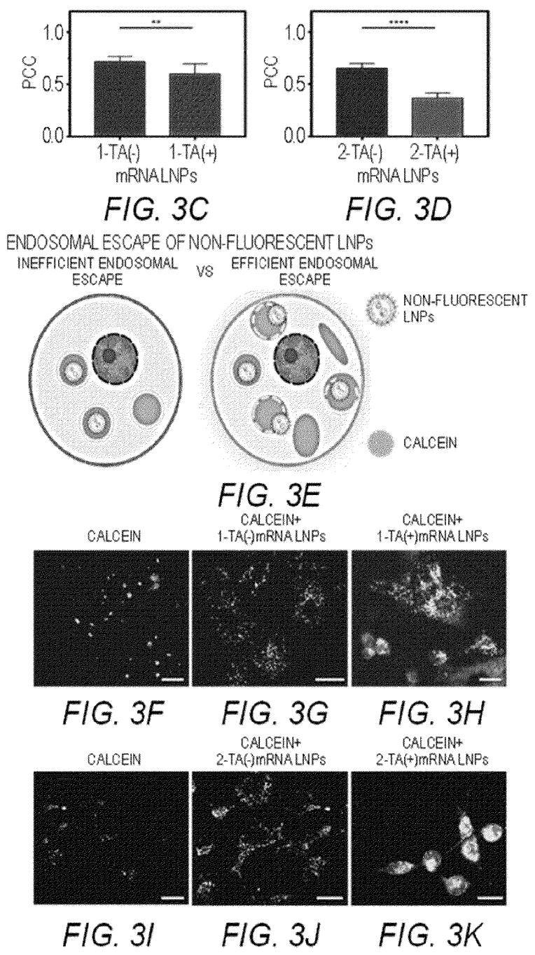

Different criteria are available for selecting the nucleic acid for use and may comprise scanning the mRNA sequence of the target, and may include empiric determination in accordance with, for example, Sui G et al., Proc. Natl. Acad. Sci. USA 99:5515-20 (2002) and may include confirmation the sequence lacks significant sequence homology with other genes as analyzed by BLAST search. Additional approaches may comprise any accessible site in endogenous mRNA that can be targeted for degradation by synthetic oligodeoxyribonucleotide/RNase H method (see, e.g., Lee N S et al., Nature Biotechnol. 20:500-05 (2002)). RNAi treatment may comprise miRNA or siRNA, or a pre-miRNA which is processed by Dicer to form a miRNA. The RNAi may also comprise a dsRNA or shRNA which is processed by Dicer to form a siRNA. The polynucleotides may comprise one or more modifications to suppress innate immune activation, enhance activity and specificity, and reduce off-target induced toxicity. Example teachings can be found, for example at Provost et al., E.M.B.O.J., 2002 Nov. 1; 21 (21): 5864-5874; Tabara et al., Cell 2002 Jun. 28; 109 (7): 861-71; Martinez et al., Cell 2002 Sep. 6; 110 (5): 563; Hutvagner & Zamore, Science 2002, 297:2056. In certain embodiments, a single-stranded RNAi agent disclosed herein can comprise substitutions, or modifications, including chemically modified nucleotides, and non-nucleotides which may include incorporation in the backbone, sugars, bases, or nucleosides. In an example, siRNA may comprise dual ribose modifications, including 2′,4′- and 2′,5′-modifications, 5′-E/Z-vinylphosphonate, and northern methanocarbacyclic (NMC) modifications. See, Gangopadhyay, RNA Biol. 2022 January; 19 (1): 452-467; doi: 10.1080/15476286.2022.2052641. The use of substituted or modified single-stranded RNAi agents can be designed to have an increased half-life in a subject. Furthermore, certain substitutions or modifications can be used to improve the bioavailability of single-stranded RNAi agents by targeting particular cells or tissues or improving cellular uptake of the single-stranded RNAi agents. Exemplary modifications and locations within a RNAi polynucleotide are described in Hu et al. “Therapeutic siRNA: State of the Art” Signal Transduction and Targeted Therapy 5, Article number 100 (2020), incorporated herein by reference, see, e.g., FIGS. 2 and 3, specifically for its teachings of modifications.

The RNAi molecule may decrease the mRNA level in a cell for a target gene by at least about 5%, about 10%, about 20%, about 30%, about 40%, about 50%, about 60%, about 70%, about 80%, about 90%, about 95%, about 99%, or about 100% of the mRNA level found in the cell without the presence of the miRNA or RNA interference molecule

The cargo may include a gene modulating agent, including gene editing systems, or a component thereof. Gene editing systems may comprise a CRISPR system, a zinc finger nuclease system, a meganuclease, or a TALE system. A CRISPR-Cas system can comprise a Class 1 or Class 2 CRISPR-Cas system, which may comprise a guide sequence engineered to specifically bind a polynucleotide of interest. A polynucleotide encoding the CRISPR-Cas polypeptide, a guide sequence designed to complex with the CRISPR-Cas polypeptide at a target of interest, or both components, can be utilized as cargo, delivered in the same nanoparticle or in different nanoparticles. As such, a ribonucleoprotein comprised of a Cas protein and a guide polynucleotide can be delivered. The CRISPR-Cas system that can be used to modify a target polynucleotide of the present invention described herein can be a Class 1 CRISPR-Cas system. Class 1 CRISPR-Cas systems are divided into types I, II, and IV. Makarova et al. 2020. Nat. Rev. 18:67-83, particularly as described in FIG. 1. Type I CRISPR-Cas systems include Types I-A, I-B, I-C, I-D, I-E, I-F1, I-F2, I-F3, and IG; Type III CRISPR-Cas systems can be Types III-A, III-B, III-C, III-D, III-E, and III-F; which can contain a Cas10 that can include an RNA recognition motif called Palm and a cyclase domain that can cleave polynucleotides; Type IV CRISPR-Cas systems include Types IV-A, IV-B, and IV-C. Class 2 systems comprise a single, large, multi-domain effector protein and can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Class 2, Type II systems include II-A, II-B, II-C1, and II-C2; Type V systems include V-A, V-B1, V-B2, V-C, V-D, V-E, V-F1, V-F1 (V-U3), V-F2, V-F3, V-G, V-H, V-I, V-K (V-U5), V-U1, V-U2, and V-U4. Class 2, Type VI systems include VI-A, VI-B1, VI-B2, VI-C, and VI-D. Design of guides for targeting a nucleic acid for modification is known in the art, see, e.g., IDTdna.com and Synthego.com for guidance on custom guide RNAs. Reduction of off-target effects can be tailored using programs such as GUIDE-seq for the design of guide sequences for a desired target. See, e.g., Malinin, et al., Nature Protocols, 16, 5592-5615 (2012).

TALEN based gene editing is also contemplated and can be used in in vivo applications. See, S Becker, J Boch-Gene and Genome Editing, 2021. Zinc finger nuclease editing can also be utilized, and further modified to ensure high-precision gene editing. See, e.g., Conway et al., Molecular Therapy, 27:4, 10 Apr. 2019, Pages 866-877; Paschon et al. Nature Comm, 10:1133 (2019). Similarly, editing can be made by meganucleases, characterized by a large recognition site of 12 to 40 based pairs of a double-stranded DNA sequence. See, e.g., U.S. Pat. Nos. 8,119,381, 10,273,524. Gene editing tools are well known in the art, with advantages and comparison of the tools that can be considered for the desired application. Rahim et al., Int'l J. of Innovative Science and Research Tech., 6:8 (2021), incorporated herein by reference.

### Transposase

Transposases may be used with the methods of the present invention. Transposases include those comprising RNase H-like nuclease domains, such as Tn5, MuA, Mos1, Hermes, Serine and Tyrosine recombinases, including CTnDOT, Tn916, IS607 and TnpX, transposases comprising an HUH domain, including TnpA of IS91 or ISHp608, and helitron transposases, which can be as detailed in International Patent Publication WO2022056309, page 26, line 26-page 27, line 17, specifically incorporated by reference. See also nuclease guided transposase as described in WO2022150651 (DNA nuclease guided Transposases systems, Tn7-like transposition proteins with a Cas12k protein), WO2022147321 (Type I-B CRISPR Associated Transposase systems), WO2022076830 (Type I CRISPR Associated transposase systems), WO2021257997 (CAST); Li, et al., Int. J. Mol. Sci. 2020, 21 (21), 8329; doi: 10.3390/ijms21218329 (Tn5 transposase in applied genomic research).

### Therapeutic Agents

Therapeutic agents that can be used as cargo with the nanoparticles can comprise modulating agents, for example, small molecules such as chemotherapeutic agents, anti-oncogenic agents, anti-microbial agents, peptides, proteins (enzymes, antibodies, peptidic hormones), non-peptidic hormones, other pharmaceutically active substances, and the like.

Examples of the protein or peptide can include hormones such as growth hormones, growth factors, cytokines, tumor necrosis factors, growth hormone releasing factors, steroid sparing agents such as cyclosporine, thyroid hormones, adrenaline, insulin, cortisol, estrogen and progesterone. Example enzymes can include transferases, hydrolases, lyases, isomerases, ligases, oxidoreductases, and translocases.

Anti-microbials such as antibiotics, antivirals, antifungals and antiparasitics can be delivered in the nanoparticles of the present invention. Accordingly, the antimicrobials can be used to prevent and/or treat infections in a subject.

Examples of a chemotherapeutic agent include without limitation: alkylating agents (e.g., which may include doxorubicin, cyclophosphamide, estramustine, carmustine, mitomycin, bleomycin and the like); antimetabolites (e.g., which may include 5-fluoro-uracil, capecitabine, gemcitabine, nelarabine, fludarabine, methotrexate and the like); platinating agents (e.g., which may include cisplatin, oxaliplatin, carboplatin and the like); topoisomerase inhibitors (e.g., which may include topotecan, irinotecan, etoposide and the like); tubulin agents (e.g., which may include paclitaxel, docetaxel, vinorelbine, vinblastine, vincristine, other taxanes, epothilones, and the like); signaling inhibitors (e.g., kinase inhibitors, antibodies, farnesyltransferase inhibitors, and the like); and other chemotherapeutic agents (e.g., tamoxifen, anti-mitotic agents such as polo-like kinase inhibitors or aurora kinase inhibitors, and the like).

Antibodies can be utilized in the methods of the invention. An antibody, or antigen binding fragment thereof, including polyclonal and monoclonal antibodies can be utilized as cargo. The term “antibody fragment” refers to a portion of an immunoglobulin, often the hypervariable region and portions of the surrounding heavy and light chains that displays specific binding affinity for a particular target, typically a molecule. A hypervariable region is a portion of an immunoglobulin that physically binds to the polypeptide target. An antibody fragment thus includes or consists of one or more portions of a full-length immunoglobulin retaining the targeting specificity of the immunoglobulin. Such antibody fragment may for instance lack at least partially the constant region (Fc region) of the full-length immunoglobulin. In some embodiments, an antibody fragment is produced by digestion of the full-length immunoglobulin. An antibody fragment may also be a synthetic or recombinant construct that contains one or more parts of the immunoglobulin or immunoglobulin chains (see e.g., HOLLIGER, P. and Hudson, J. Engineered antibody fragments and the rise of single domains. Nature Biotechnology 2005, vol. 23, no. 9, p. 1126-1136). Examples of an antibody fragment include, but are not limited to, an scFv, a Fab, a Fv, a Fab′, a F(ab′) 2 fragment, a dAb, a VHH, a nanobody, a V(NAR) or a so-called minimal recognition unit.

Another aspect of the invention is a method of treating or preventing a disease in a subject in need thereof, the method comprising administering to the subject a therapeutically or prophylactically effective amount of one or more nanoparticles comprising a cargo, thereby treating or preventing the disease. In some embodiments, the method comprises administering to a subject an effective amount of one or more nanoparticles comprising a cargo to decrease the risk of developing a disease or to reduce the severity of disease should it occur. In some embodiments, the administering is to reduce the likelihood that the disease will occur, delay the onset of the disease and/or reduce the severity of the diseases including number of symptoms, severity of symptoms and/or length disease course. In some embodiments, the method comprises administering to a subject an effective amount of one or more nanoparticles comprising a cargo to a subject who has a disease or is at increased risk of developing a disease relative to a member of the general population.

In some embodiments, the methods treat or prevent a disease, disorder, or condition by introducing a cargo, thereby modulating the expression of a gene. An example application includes vaccine therapies, cancer treatments (e.g., melanoma), infection disease therapies, and gene editing. The administration of a nanoparticle comprising a cargo may increase or enhance expression or reduce or silence gene expression.

Another aspect of the invention is methods to detect, diagnose, or monitor a disease, disorder, or condition. Methods of detection can be in vitro and can comprise obtaining a sample from the subject. Suitable samples may comprise blood, serum, plasma, brain homogenate, interstitial fluid, cerebral spinal fluid, and/or exocrine gland secretion, and enriched forms thereof. According to one embodiment, the sample is a biological sample from a subject in need thereof. The biological sample can be from a subject that requires diagnosis of disease, and/or monitoring of the effectiveness of a treatment.

Methods of detection can be in vivo and can comprise administering a nanoparticle comprising an imaging agent or detectable label to the subject. The nanoparticle may comprise a labeled biomarker, which includes nucleic acids, proteins, metabolites and reaction products thereof. Such biomarkers also encompass the mutations, variants, modifications, fragments, and polymorphisms of said biomarkers. Preferably, the subject requires diagnosis of, or is suspected of having, a disease or disorder, and optionally monitoring of the effectiveness of a treatment or progression of a disease. Accordingly, the nanoparticles comprising a cargo such as a biomarker, label or imaging agent are useful in methods of diagnosing, prognosing and/or staging a disease or disorder in a subject by detecting a first level of expression, activity and/or function of one or more biomarker and comparing the detected level to a control of level wherein a difference in the detected level and the control level indicates.

An aspect of the present invention is a method of transferring or delivering a cargo to a cell in vitro. The nanoparticle comprising a cargo may be introduced, e.g., by contact, into the cells at the appropriate cargo dosage suitable for the particular target cells. In some embodiments, the nanoparticle is designed for use in an in vitro application and may comprise a polyphenol with a lower molecular weight, as described elsewhere herein. The amount of nanoparticle to administer can vary, depending upon the target cell type and number, and the particular nanoparticle and cargo, and can be determined by those of skill in the art without undue experimentation.

Methods of making a cargo-loaded nanoparticle are provided, comprising: adding an organic phase comprising a polyphenol, a sterol, a PEG lipid, an ionizable lipid and a phospholipid to an aqueous phase comprising a cargo to make a mixture. The cargo and nanoparticle components can be selected to optimize electrostatic interactions between the cargo and the nanoparticle composition. The ratios of the polyphenol lipid nanoparticle components can be further tuned to optimize interactions with the cargo. In an aspect, one or more of the amounts or compositions of the ionizable lipid, phospholipid, sterol, PEG lipid, and/or polyphenol components can be adjusted for optimization.

Methods of increasing endosomal escape of a LNP delivered to a cell are provided, comprising including a polyphenol in the LNP.

A further aspect of the invention is a method of treating subjects in vivo, comprising administering to a subject nanoparticles comprising one or more cargo, which may be further comprised in a pharmaceutically acceptable carrier, wherein the pharmaceutical composition is administered in a therapeutically effective amount. Administration of the nanoparticles of the present invention to a human subject or an animal in need thereof can be by any means known in the art for administering compounds. In some embodiments, the nanoparticle is designed for use in an in vivo application, and may comprise a polyphenol with a higher molecular weight, as described elsewhere herein.

Administration may preferably be by intravenous or intramuscular administration. In an example embodiment, the method comprises administering a nanoparticle comprising a therapeutic as a vaccine, wherein the administration is performed intramuscularly. In an example embodiment, the method comprises administering a nanoparticle comprising a therapeutic for treatment or prevention of a disease of disorder comprising administering the nanoparticle intravenously.

In an aspect, the delivery dose is at 0.01 to 10.00 mg mL⁻¹, 0.05 to 5 mg mL⁻¹, or between about 0.1 and 1.0 mg mL⁻¹. In certain embodiments, disclosed compounds are administered at dosage levels greater than about 0.001 mg/kg, such as greater than about 0.01 mg/kg or greater than about 0.1 mg/kg. For example, the dosage level may be from about 0.001 mg/kg to about 50 mg/kg such as from about 0.01 mg/kg to about 25 mg/kg, from about 0.1 mg/kg to about 10 mg/kg, or from about 1 mg/kg to about 5 mg/kg of subject body weight per day, one or more times a day, to obtain the desired therapeutic effect. It will also be appreciated that dosages smaller than about 0.001 mg/kg or greater than about 50 mg/kg (for example about 50-100 mg/kg) can also be administered to a subject.

Non-limiting examples of formulations of the invention include those suitable for intravenous administration of the nanoparticles. Oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor) are also envisioned. The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site, or intramuscular administration with muscle specific promoters). In some embodiments, the formulation can be delivered locally to ischemic tissue.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

In one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See, e.g., U.S. Pat. No. 4,501,729. Aerosols of the particles of the invention can likewise be produced with any solid particulate medicament aerosol generator, by techniques known in the pharmaceutical art.

### EXAMPLES

### Example 1. Tannic Acid Lipid Nanoparticles

Even though lipid nanoparticles (LNPs) can deliver messenger RNA (mRNA) payloads into cells, their efficiency is often limited by endosomal trapping which prevents RNA payloads from acting therapeutically. Improving the percentage of RNA LNPs that can escape from endosomes and enter the cytoplasm is therefore an area of active research interest that could lead to improved safety profiles and reduced manufacturing costs of mRNA drugs.

Whereas some strategies to improve endosomal escape focus on manipulating the biological pathways of cells, Applicant instead hypothesized that the endosomal escape of COVID19 inspired mRNA LNPs could be improved by incorporating endosome-destabilizing small molecules within these formulations.^{122,257} More specifically, it was hypothesized that this goal could be accomplished by incorporating polyphenols into mRNA LNP formulations inspired by the Moderna and Pfizer/BioNTech COVID19 vaccines (FIG. 1). Briefly, polyphenols are a class of naturally occurring compounds that are characterized by multiple hydroxyl groups attached to aromatic carbons in a given molecule.^{[29-31]} This unique structural feature imparts polyphenols with a high affinity to interact with biomolecules such as proteins, polysaccharides, and nucleic acids, a property that Applicant hoped to leverage to incorporate polyphenols within mRNA LNPs to improve endosomal escape.^{[32-36}]

Applicant began the study by performing a structural evaluation of polyphenolic compounds to identify a lead candidate for incorporation within the mRNA LNPs. To maximize the clinical relevance of the work, Applicant sought to identify polyphenols that: i. are naturally occurring; ii. are considered “generally recognized as safe” (GRAS) by the FDA; iii. are available commercially; iv. have multiple hydrogen bond donors and acceptors to complex the mRNA backbone and bases; v. are of discrete molecular weight, vi. are soluble at concentrations required for mRNA LNP formulation, and vii. are well tolerated. After careful evaluation, tannic acid (TA) was ultimately selected as the lead polyphenolic candidate for incorporation within mRNA LNPs. In addition to boasting structural features that can interact with biomolecules including 10 aromatic rings, 25 phenolic hydroxyl groups, and a sugar core, TA also met all the aforementioned design criteria^{[37,38]}. Moreover, the effects that TA incorporation has on the formulation, characterization, endosomal escape, and efficacy of mRNA LNPs has not yet been explored which further piqued the interest in undertaking this approach.

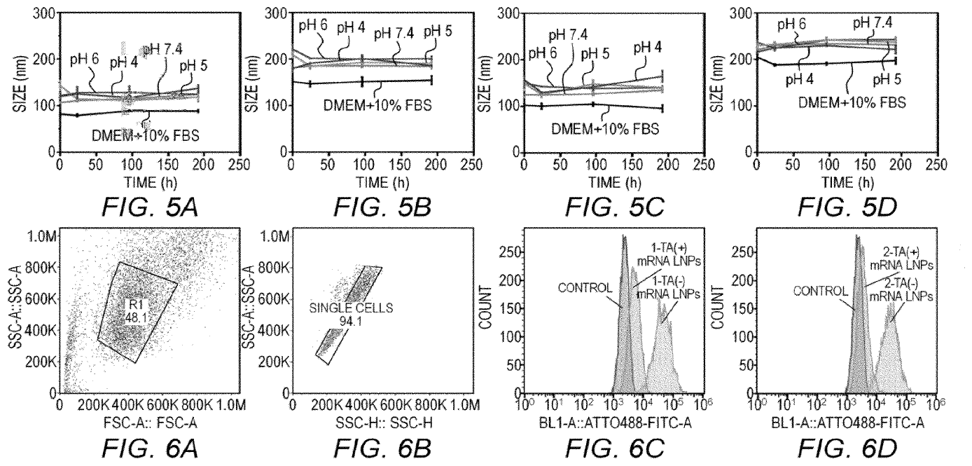

Having identified TA as a lead candidate, efforts were then focused on formulating TA(+) mRNA LNPs. Briefly, microfluidic mixing was used to formulate each TA(+) mRNA LNP by mixing an ethanol phase [containing TA (a polyphenol—to improve the endosomal escape of the LNPs),^{[39]} cholesterol (a sterol—to improve the stability of the LNPs),^{[40,41]} C14-PEG-2000 (a PEG lipid—to prevent particle aggregation and reduce non-specific uptake),^{[42]} SM-102 or ALC-0315 (ionizable lipids from the Moderna and Pfizer/BioNTech COVID19 LNP formulations respectively—to complex the mRNA),^{[43]} and DOPE (a phospholipid—to improve the bilayer structure of the LNPs)]^{[44]} with a citrate buffered aqueous phase containing mRNA encoding for firefly luciferase (FLuc) (FIGS. 2A-2B). To isolate the effect that TA has on the properties of mRNA LNPs, formulations that did not contain TA [denoted as TA(−) mRNA LNPs] were also prepared. The size distribution, mRNA encapsulation efficiencies, and zeta potentials were determined for each of these 4 formulations which were denoted as 1-TA(+) and 2-TA(+) as well as 1-TA(−) and 2-TA(−) (FIGS. 2C-2E). In analyzing these data, several trends were observed. First, dynamic light scattering revealed that the size distribution of TA(+) mRNA LNPs was larger than the size distribution of TA(−) mRNA LNPs (ex. formulations 1-TA(+)=188.5±10.5 nm vs. 1-TA(−)=109.5±1.5 nm; 2-TA(+)=221.5±2.8 nm vs. 2-TA(−)=125.3±0.6 nm, FIG. 2C and Table 1). Further, the encapsulation efficiency of FLuc mRNA within TA(+) mRNA LNPs was higher than in TA(−) mRNA LNPs (ex. 1-TA(+)=84.0±0.4% vs. 1-TA(−)=68.6±0.6%; 2-TA(+)=85.1=0.6% vs. 2-TA(−)=64.7±1.5%, FIG. 2D). Charge-reversal studies also demonstrated that the TA(+) mRNA LNPs and TA(−) mRNA LNPs exhibited a positive-to-negative zeta potential shift around pH 6 (FIG. 2E), and stability studies using dynamic light scattering suggested that there was no aggregation or disassembly of the TA(+) mRNA LNPs or TA(−) mRNA LNPs at 4° C. for up to one week when stored in diverse pH conditions (pH=4.0, 5.0, 6.0, 7.4) or in Dulbecco's modified Eagle medium (DMEM) supplemented with 10% fetal bovine serum (FBS) (FIGS. 5A-5D).

To investigate the ability to use the TA(+) LNPs to deliver mRNA payloads into cells, in vitro FLuc expression and cellular viability studies were performed on Madin-Darby Canine Kidney (MDCK) cells. MDCK cells were selected because some RNA drugs accumulate in the kidneys when administered without a carrier^{[45,46]} For FLuc expression and cellular viability studies, MDCK cells were treated with equivalent doses of either 1-TA(+) mRNA LNPs or 2-TA(+) mRNA LNPs; to isolate the effect that TA imparts on FLuc expression and cellular viability, 1-TA(−) mRNA LNPs and 2-TA(−) mRNA LNPs were also evaluated. After 24 hours, FLuc expression per live cell for each LNP was quantified using a Bright-Glo Luciferase Assay Kit (Promega, FIG. 2F), and viability was assessed using an XTT cell proliferation assay (FIG. 2G). A collective analysis of these data sets first confirmed that TA(+) mRNA LNPs are well tolerated and also that cells treated with TA(+) mRNA LNPs express FLuc protein in a statistically significant fashion relative to cells treated with PBS alone. Further, TA incorporation did not negatively impact FLuc expression (ex. 1-TA(+) and 1-TA(−) express statistically identical levels of FLuc; 2-TA(+) and 2-TA(−) also express statistically identical levels of FLuc, FIG. 2F). DSPC incorporation in the mRNA LNPs resulted in statistically lower levels of luciferase expression in treated cells (FIG. 11); accordingly, mRNA LNP formulations based upon DOPE were elected for the remainder of the work.

To further characterize the in vitro delivery of the TA(+) mRNA LNPs, cellular association studies were also performed on MDCK cells to quantify percentage of cells that interact with the designed particles. In brief, fluorescence associated cell sorting (FACS) was performed on MDCK cells treated with fluorescently labelled (ATTO-488) analogues of the 1-TA(+) and 2-TA(+) mRNA LNP formulations; to isolate the effect that TA imparts on cellular association, FACS was also performed on fluorescently labelled (ATTO-488) analogues of the 1-TA(−) mRNA LNPs and 2-TA(−) mRNA LNPs. An analysis of this FACS data revealed that TA incorporation within these mRNA LNPs decreased cellular association in a statistically significant fashion (ex. cellular association of 1-TA(+)=57.7±0.9% vs. cellular association of 1-TA(−)=99.4±0.4%; cellular association of 2-TA(±)=22.2±1.0% vs. cellular association of 2-TA(−)=98.7±1.6%, FIG. 2H and FIG. 6). Despite this statistically significant decrease in cellular association in TA(+) vs TA(−) mRNA LNPs, the incorporation of TA in these same mRNA LNPs did not impact the expression nor the viability of FLuc in cells treated with TA(+) vs. TA(−) mRNA LNPs as was observed in FIG. 2F and FIG. 2G, respectively.

Given that theTA(+) mRNA LNPs had exhibited statistically equivalent levels of luciferase expression despite lower cellular association, it was speculated that the TA(+) mRNA LNPs may have improved endosomal escape properties due to the incorporation of TA. To evaluate this hypothesis, an endosomal escape assay was developed where the location of fluorescently labelled versions of the TA(+) mRNA LNPs in cells was tracked (FIG. 3A). Briefly, confocal imaging was performed on MDCK cells [whose endosomes were stained with Lysotracker Deep Red and whose nuclei were stained with Hoechst 33342] that were treated with fluorescently labelled [ATTO-488] analogues of the 1-TA(+) mRNA LNPs and 2-TA(+) mRNA LNP formulations; to isolate the effect that TA imparts on endosomal escape, confocal imaging was also performed on MDCK cells [whose endosomes were stained with Lysotracker Deep Red and whose nuclei were stained with Hoechst 33342] that were treated with fluorescently labelled [ATTO-488] analogues of the 1-TA(−) mRNA LNPs and 2-TA(−) mRNA LNPs (FIG. 3B). Quantification of the Pearson Correlation Coefficient [PCC, a quantifiable metric of endosomal escape that is defined as the ratio of the total number of yellow dots (corresponding to overlap between red endosomes with green dots) divided by the total number of dots (corresponding to the sum of yellow and green dots)]^{[45]} was performed on cells treated with the 1-TA(+) mRNA LNPs, 1-TA(−) mRNA LNPs, 2-TA(+) mRNA LNPs, and 2-TA(−) mRNA LNPs (FIGS. 3C, 3D). An analysis of these PCC values revealed that both TA(+) mRNA LNPs had lower PCC values (i.e., more endosomal escape) than their corresponding TA(−) mRNA LNP analogues [ex. PCC of 1-TA(+)=0.60±0.04 vs. PCC of 1-TA(−)=0.72±0.05 (FIG. 3C); PCC of 2-TA(+)=0.37±0.05 vs. PCC of 2-TA(−)=0.66±0.05 (FIG. 3D)]. Taken in full, this PCC data further suggests that TA incorporation in the TA(+) mRNA LNPs positively impacts endosomal escape as measured by a quantitative comparison between mRNA LNPs entrapped inside and outside of endosomal compartments.

To further validate this finding, a second endosomal escape experiment was developed where cells that contained fluorescent molecules within their endosomes were imaged after treatment with the original non-fluorescent TA(+) mRNA LNPs (FIG. 3E). Briefly, MDCK cells whose endosomal compartments contained calcein (a membrane impermeable dye that remains entrapped within intact endosomes but becomes distributed throughout cells if endosomal membranes are ruptured)^{[46]} were imaged 24 hours after treatment with either the 1-TA(+) mRNA LNPs, 1-TA(−) mRNA LNPs, 2-TA(+) mRNA LNPs, or 2-TA(−) mRNA LNPs; images of calcein-containing MDCK cells that were not treated with mRNA LNPs were also acquired as a control (FIGS. 3F-3K). Analysis of these images reveals that cells treated with TA(+) mRNA LNPs exhibit a broader distribution of calcein throughout the cell than was observed in cells that had been treated with TA(−) mRNA LNPs or untreated cells [ex. greater calcein distribution was observed throughout cells treated with 1-TA(+) mRNA LNPs (FIG. 3H) than cells treated with 1-TA(−) mRNA LNPs (FIG. 3G) or untreated cells (FIG. 3F); greater calcein distribution was also observed throughout cells treated with 2-TA(+) mRNA LNPs (FIG. 3K) than cells treated with 2-TA(−) mRNA LNPs (FIG. 3J) or untreated cells (FIG. 3I)] which further supports the hypothesis that TA improves endosomal escape of these LNPs.

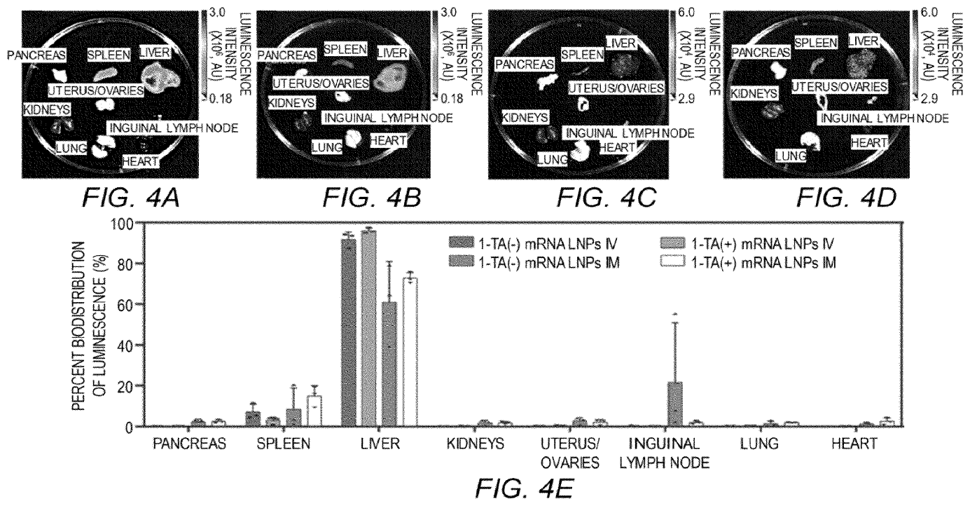

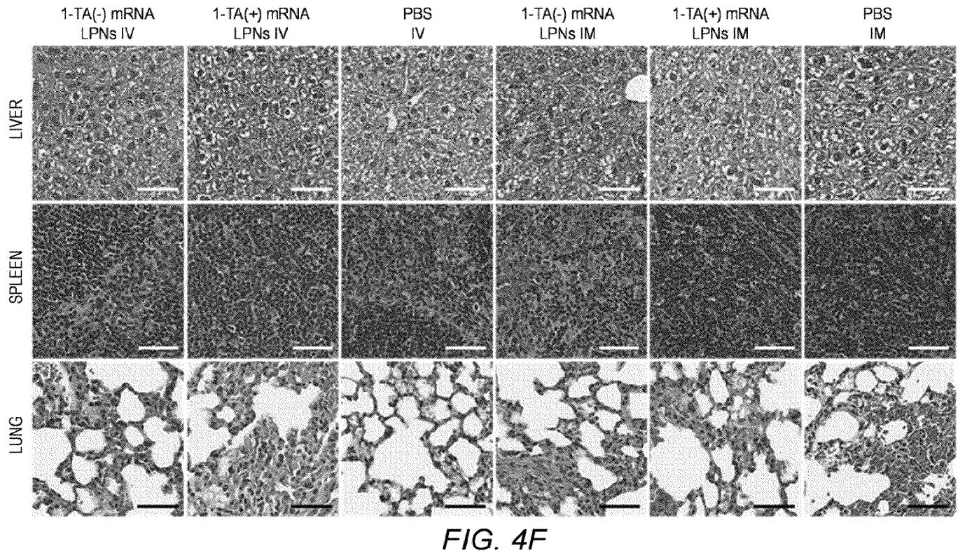

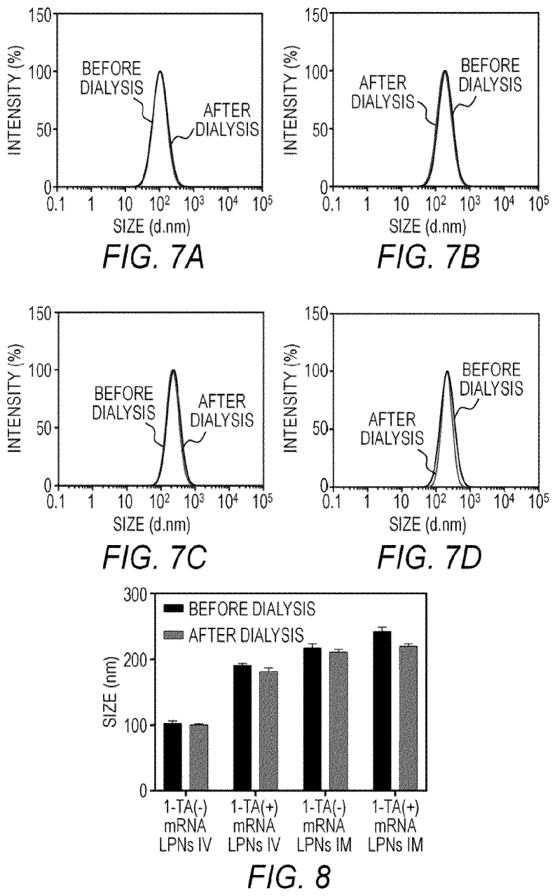

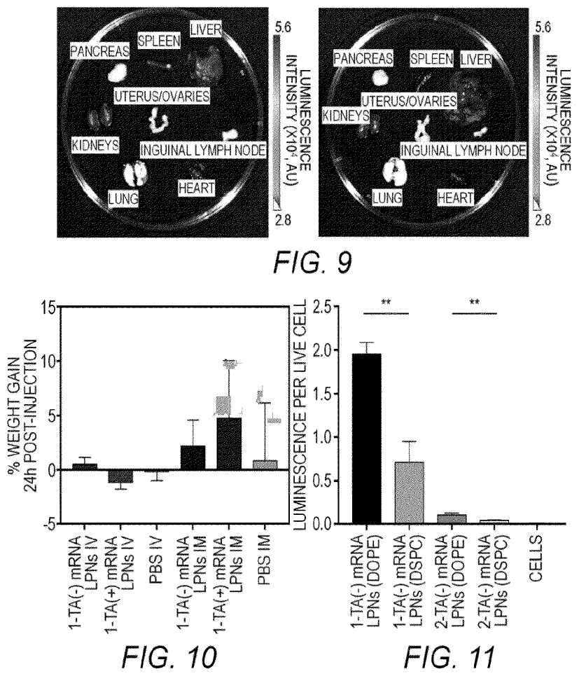

To further investigate the ability to use the TA(+) LNPs as mRNA drug carriers, the study was concluded by investigating the ability of 1-TA(+) mRNA LNPs to deliver mRNA in vivo. The Applicant elected to study the 1-TA(+) mRNA LNPs in vivo given their favorable cellular viability and high expression of FLuc in vitro relative to the 2-TA(+) system (FIGS. 4A-4F, Table 2 and FIGS. 7A-7D, 8). Intravenous (IV) and intramuscular (IM) routes of administration were explored given their utility for therapeutic and vaccination applications of mRNA LNPs respectively. Briefly, 1-TA(+) mRNA LNPs were administered into female Black 6 mice using either an intravenous (IV) or intramuscular (IM) dosing regimen; to isolate the effect that TA imparts on in vivo activity, the 1-TA(−) mRNA LNPs were also administered into a different treatment group of mice, and phosphate buffered saline was administered to yet another cohort of mice to serve as a negative control. Each mouse's organs were harvested and imaged ex vivo using an IVIS imaging system 24 hours post injection (FIGS. 4A-4D) and the total luminescence per organ was also quantified (FIG. 4E). When administered IV, the 1-TA(+) mRNA LNPs resulted in predominant luciferase expression in the liver and less expression in the spleen (FIG. 4B). IM injections of the 1-TA(+) mRNA LNPs also resulted in a similar expression profile but with reduced overall intensity levels in luciferase expression (FIG. 4D). A similar biodistribution profile was also observed for 1-TA(−) mRNA LNPs that were administered intravenously or intramuscularly which suggests that TA incorporation does not significantly impact the expressed FLuc biodistribution of these mRNA LNPs. No luminescence was detected in the organs of mice treated with PBS (FIG. 9). Further, histological analysis (FIG. 4F) and weight gain studies (FIG. 10) indicated that the 1-TA(+) LNPs were able to deliver mRNA payloads in a well-tolerated fashion.

In summary, TA(+) LNPs are reported as an effective drug delivery system for mRNA delivery in vitro and in vivo. In addition to validating TA(+) LNPs as a viable delivery platform for mRNA payloads, the study also isolates the effects that TA incorporation imparts on properties including the size, charge, encapsulation efficiency, mRNA encoded protein expression, viability and cellular association of the mRNA LNPs using a side-by-side comparison approach. Further, this study also explores two approaches using confocal imaging to study the endosomal escape of mRNA LNPs, both of which highlight that TA incorporation within LNPs positively impacts their endosomal escape. Without being bound by theory, this enhancement is possibly influenced by the ability of TA to form molecular interactions with biomolecules in the cell and endosomal membrane, and further study will continue to investigate these interactions to complement the endosomal escape studies. Future studies will explore the potential of TA(+) LNPs to deliver alternative nucleic acid cargoes to mRNA including siRNA and pDNA. In summary, by studying the effects of TA incorporation on LNP properties and delivery, this report suggests that TA(+) LNPs may be a useful platform for the delivery of mRNA payloads in vitro and in vivo which may be useful in the development of next-generation therapeutics for the study and prevention of disease.

TABLE 1
Mean size, PDI and zeta potential of 1-TA(−) mRNA LNPs,
1-TA(+) mRNA LNPs, 2-TA(−) mRNA LNPs, and 2-TA(+) mRNA
LNPs after mRNA encapsulation (The final concentration of mRNA is
0.1 mg mL⁻¹). Data are shown as the mean ± SD, N = 3.

| | | | Zeta |
|---|---|---|---|
| | Size (nm) | PDI | potential (mV) |
| 1-TA(−) mRNA LNPs | 109.5 ± 1.5 | 0.14 ± 0.03 | −4.36 ± 1.52 |
| 1-TA(+) mRNA LNPs | 188.5 ± 10.5 | 0.21 ± 0.01 | −3.83 ± 1.52 |
| 2-TA(−) mRNA LNPs | 125.3 ± 0.6 | 0.17 ± 0.02 | −6.27 ± 3.33 |
| 2-TA(+) mRNA LNPs | 221.5 ± 2.8 | 0.07 ± 0.02 | −4.34 ± 0.73 |

TABLE 2
Encapsulation efficiencies of 1-TA(−) mRNA LNPs
IV, 1-TA(+) mRNA LNPs IV, 1-TA(−) mRNA LNPs
IM and 1-TA(+) mRNA LNPs for intravenous and intramuscular
administration. IV and IM doses were administered at 0.15 mg
mL⁻¹ and 0.35 mg mL⁻¹ respectively.

| | 1- | 1- | 1- | 1- |
|---|---|---|---|---|
| | TA(−) | TA(+) | TA(−) | TA(+) |
| | mRNA | mRNA | mRNA | mRNA |
| | LNPs | LNPs | LNPs | LNPs |
| | IV | IV | IM | IM |
| mRNA | 67.6% ± | 82.7% ± | 37.8% ± | 62.7% ± |
| Encapsulation | 0.4% | 0.5% | 1.3% | 0.9% |
| efficiency | | | | |

### Conclusion

Applicant reports herein example polyphenol mRNA LNPS, tannic acid mRNA LNPs [TA(+) mRNA LNPs], as an effective delivery platform for the delivery of mRNA payloads in vitro and in vivo. The formulation, characterization, and stability of the TA(+) mRNA LNPs is described; two confocal microscopy approaches are then utilized to quantify their endosomal escape; and lastly the biodistribution and tolerability of the TA(+) mRNA LNPs is evaluated in mice following intravenous and intramuscular dosing regimens. To isolate the effect that TA imparts on these properties, mRNA LNPs that do not contain TA [TA(−) mRNA LNPs] are evaluated side-by-side in each experiment. These findings suggest that TA can improve the endosomal escape of mRNA LNPs, and more broadly, highlight the potential for TA(+) LNPs to deliver mRNA payloads in vitro and in vivo for the prevention and treatment of disease.

### Experimental Section

### Materials

Cholesterol, tannic acid, phosphate-buffered saline (PBS, pH 7.4), sodium citrate, citrate acid, Dulbecco's phosphate-buffered saline (DPBS), penicillin-streptomycin, ethanol phenazine methosulfate (PMS), Triton X-100, and ATTO-488 DOPE, were purchased from Sigma-Aldrich. 1,2-dioleoyl-sn-glycero-3-phosphoethanolamine (DOPE) and 1,2-dimyristoyl-sn-glycero-3-phosphoethanolamine-N-[methoxy-(polyethyleneglycol)-2000] (ammonium salt) (C14-PEG-2000) were purchased from Avanti. Moderna SM-102 Lipids and Pfizer ALC-0315 lipids were bought from Broadpharm. Dulbecco's modified Eagle's medium (DMEM), Fetal Bovine Serum (FBS), LysoTracker Deep Red, 2,3-Bis [2-methoxy-4-nitro-5-sulfophenyl]-2H-tetrazolium-5-carboxyanilide inner salt (XTT), trihydrochloride (Hoechest 33342, 10 mg mL⁻¹), calcein (high purity) were purchased from Life Technologies. Firefly luciferase mRNA was obtained from TriLink Biotechnologies. Quant-iT RiboGreen RNA assay kit was purchased from Thermo Fisher Scientific. Bright-Glo Luciferase assay system and VivoGlo Luciferin were obtained from Promega. Ultrapure water (Milli-Q) with a resistivity greater than 18.2 M (2·cm was used in all experiments and obtained from a three stage Millipore Milli-Q Plus 185 purification system. All chemicals were used without further purification.

### Synthesis of Tannic Acid Based Lipid Nanoparticles

Ethanol phase was prepared by mixing ionizable lipid, dioleoyl-sn-glycero-3-phosphoethanolamine (DOPE), cholesterol, 1,2-dimyristoyl-sn-glycero-3 phosphoethanolamine-N-[methoxy-(polyethyleneglycol)-2000] (ammonium salt) (C14-PEG-2000), and tannic acid at a molar ratio of 47:9:40:2:2 for 1-TA(+) mRNA LNPs and 44:9:40:2:5 for 2-TA(+) mRNA LNPs. Aqueous phase was made by dissolving the FLuc mRNA at a weight ratio of 10:1 in 10 mM citrate buffer (pH 3). Then, the ethanol and aqueous phases were mixed microfluidically at a ratio of 1:3 at a final FLuc mRNA concentration of 0.1 mg mL⁻¹. Finally, the synthesized mRNA encapsulated TA LNPs were dialyzed against 1×PBS in a 20,000 MWCO cassette at 4° C. cold room for 3 h followed by storage at 4° C. prior for further use.

### Characterization

Size distribution profiles, PDIs, zeta potentials and stability of 1-TA(−) mRNA LNPs, 1-TA(+) mRNA LNPs, 2-TA(−) mRNA LNPs, and 2-TA(+) mRNA LNPs in different pH buffers were recorded using a NanoBrook 90 Plus Zeta (Brookhaven, USA). The FLuc mRNA encapsulation efficiency of TA(−) mRNA LNPs and TA(+) mRNA LNPs was measured by Quant-iT RiboGreen RNA assay kit a SpectraMax microplate reader (Molecular Device, USA) with 480 nm excitation wavelength and 520 nm emission wavelength.

### In Vitro FLuc Expression

MDCK cells were seeded at a density of 1×10⁴ cells per well in a 96 well plate at 37° C. overnight. The cell media was then aspirated and the cells were then treated with 100 μL of a 500 ng mL⁻¹ dose of the 1-TA(−) mRNA LNPs, 1-TA(+) mRNA LNPs, 2-TA(−) mRNA LNPs, or 2-TA(+) mRNA LNPs where the mRNA encoded for the protein firefly luciferase (FLuc). After 24 hours, the luciferase expression per live cell was quantified using a Bright-Glo Luciferase assay system using a SpectraMax microplate reader with luminescence function.

### Cell Viability by XTT Assay

The activated XTT reagent was prepared by mixing 9 mL of 0.2 mg mL⁻¹ XTT in DMEM and 22.5 μL of 0.6 mg mL⁻¹ PMS in DPBS). MDCK cells were grown in DMEM supplemented with 10% fetal bovine serum (FBS) and 1% penicillin-streptomycin at 37° C. in a 5% CO₂ incubator. MDCK cells were seeded in a 96-well Transwell plate at 1×10⁴ cells per well in 100 μL media and incubated overnight. The next day, the cell media was removed and 100 μL of fresh cell media (for cell control), 100 μL of ethanol (as a negative “kill” control to ensure the assay was properly functioning) and 100 μL of fresh media containing TA(−) mRNA LNPs and TA(+) mRNA LNPs at 500 ng mL⁻¹ of mRNA were added. After 24 h, the media was replaced with 100 μL of activated XTT media. After 3 h, the absorbance of the solution was measured using a SpectraMax microplate reader with 475 nm wavelength with a reference wavelength of 675 nm. Untreated cells containing only DMEM was used as background controls. The cell viability results were then calculated by normalizing absorbance to the untreated cells.

### Cell Association Studies

MDCK cells were seeded in a 24-well transparent well plate at a density of 8×10⁴ cells per well and incubated in complete DMEM media at 37° C. overnight to allow the cellular adhesion to the substrate. On the next day, 500 ng mL⁻¹ of mRNA encapsulated ATTO-488 labelled TA(−) mRNA LNPs and TA(+) mRNA LNPs were added to the cells followed by incubation for 24 h at 37° C. Subsequently, the cells were washed with DPBS three times and dissociated using trypsin-EDTA. Finally, the samples are analyzed on Attune Flow Cytometer (Thermofisher Scientific, USA). The cell association was assessed by using the percentage of cells with stronger fluorescence intensity than the control untreated cells.

### Assessing Endosomal Escape of Fluorescent Lipid Nanoparticles Using Confocal Microscopy

MDCK cells were seeded at a density of 4×10⁴ cells per well in u-slide 8-well coverslip slides at 37° C. overnight to allow the cellular adhesion to the substrate. On the next day, the cell media was aspirated, and 300 μL of ATTO-488 labelled TA(−) mRNA LNPs and TA(+) mRNA LNPs were introduced to the cells at a FLuc mRNA concentration of 500 ng mL⁻¹ that were then incubated for 24 h at 37° C. After the incubation, the cells were washed with DPBS three times, and 300 μL of 100 nM of LysoTracker Deep Red (that had been dissolved in prewarmed 37° C. culture media), was added. After 1 h, cells were washed with DPBS for three times and 1 μg mL⁻¹ of Hoechst 33342 was introduced to cells for 10 min to stain the nucleus. Finally, the live cell imaging was performed by confocal laser scanning microscope (CLSM) (Leica SP8X, USA) with 60× water immersion objective. The images were further processed by WCIF Image J software. Experiments were performed in triplicate and ten representative cell images (>50 cells) were used to calculate the PCC value.

Assessing Endosomal Escape of Non-Fluorescent Lipid Nanoparticles Using Confocal Microscopy with a Calcein Leakage Assay

MDCK cells were seeded at a density of 4×10⁴ cells per well in u-slide 8-well coverslip slides at 37° C. overnight. Subsequently, the cell media was removed and 180 μL of TA(−) mRNA LNPs and TA(+) mRNA LNPs were added into the cells at a FLuc mRNA concentration of 500 ng mL⁻¹ followed by adding 20 μL 1.5 mg mL⁻¹ of calcein. After 24 h incubation at 37° C., the cells were washed with DPBS three times to remove extracellular calcein, TA(−) mRNA LNPs and TA(+) mRNA LNPs. Finally, live cell imaging was performed by CLSM with 40× water immersion objective.

### Animal Studies-FLuc Expression and Histology

All animal studies were approved by the UNC Institutional Animal Care and Use Committee, were consistent with local, state, and federal regulations as applicable, and were supported within the UNC Lineberger ASC at the University of North Carolina at Chapel Hill.

For intravenous dosing studies, 1-TA(−) FLuc mRNA LNPs and 1-TA(+) FLuc mRNA LNPs were prepared to a final mRNA concentration of 0.15 mg mL⁻¹. PBS injection was used as a negative control. Each group was then injected via the tail vein of female C57 black 6 mice (Jackson Laboratory, 18-22 g) at a dose of 0.75 mg kg-1. After 24 hours, mice were injected intraperitoneally with 130 μL of D-luciferin (30 mg mL⁻¹ in PBS). After 15 min, mice were euthanized and organs (pancreas, spleen, liver, kidneys, ovaries, inguinal lymph node, lung and heart) were removed and imaged with an in-vivo imaging system (IVIS) (Perkin Elmer, Waltham, MA). Luminescence was quantified using AuRA software (Spectral instruments imaging). The liver, spleen, and lung were then fixed in 10% neutral buffered formalin after imaging and were then routinely processed to paraffin. Histological sections were evaluated using hematoxylin and eosin stain. Briefly, Paraffin embedded tissue blocks were sectioned at 5 μm onto positively charged slides. In order to proceed with histological staining, samples were first baked at 60 degrees Celsius for 60 minutes minimum and were then deparaffinized in xylene and hydrated with graded ethanols before continuing with the H&E stain. H&E stains were performed using the autostainer XL from Leica Biosystems. The sections were stained with Hematoxylin (Richard-Allen Scientific, 7211) for 2 mins and Eosin-Y (Richard-Allen Scientific, 7111) for 1 min. Clarifier 2 (7402) and Bluing (7111) solutions from Richard-Allen Scientific were used to differentiate the reaction. After staining, slides were then dehydrated and coverslipped with Cytoseal 60 (8310-4, Thermo Fisher Scientific). Histological analysis was then performed on each sample.

For intramuscular dosing studies, 1-TA(−) FLuc mRNA LNPs and 1-TA(+) FLuc mRNA LNPs were prepared to a final mRNA concentration of 0.35 mg mL⁻¹. PBS injection was used as a negative control. Briefly, 25 μL of each group was injected into the left and right quadricep (overall 50 μL of LNPs). After 24 hours, mice were injected intraperitoneally with 130 μL of D-luciferin (30 mg mL⁻¹ in PBS). After 15 min, mice were euthanized and organs (pancreas, spleen, liver, kidneys, ovaries, inguinal lymph node, lung and heart) were removed and imaged with an in-vivo imaging system (IVIS) (Perkin Elmer, Waltham, MA).

Luminescence was quantified using AuRA software (Spectral instruments imaging). The liver, spleen, and lung were then fixed in 10% neutral buffered formalin after imaging and were then routinely processed to paraffin. Histological sections were evaluated using hematoxylin and eosin stain. Briefly, Paraffin embedded tissue blocks were sectioned at 5 μm onto positively charged slides. In order to proceed with histological staining, samples were first baked at 60 degrees Celsius for 60 minutes minimum and were then deparaffinized in xylene and hydrated with graded ethanols before continuing with the H&E stain. H&E stains were performed using the autostainer XL from Leica Biosystems. The sections were stained with Hematoxylin (Richard-Allen Scientific, 7211) for 2 mins and Eosin-Y (Richard-Allen Scientific, 7111) for 1 min. Clarifier 2 (7402) and Bluing (7111) solutions from Richard-Allen Scientific were used to differentiate the reaction. After staining, slides were then dehydrated and coverslipped with Cytoseal 60 (8310-4, Thermo Fisher Scientific). Histological analysis was then performed on each sample.

### REFERENCES

[1] S. Liu, Q. Cheng, T. Wei, X. Yu, L. T. Johnson, L. Farbiak, D. J. Siegwart, Nat. Mater. 2021, 20, 701.
[2] D. Kobelt, J. Pahle, W. Walther, Methods Mol. Biol. 2022, 2521, 1.
[3] S. J. Shepherd, C. C. Warzecha, S. Yadavali, R. El-Mayta, M.-G. Alameh, L. Wang, D. Weissman, J. M. Wilson, D. Issadore, M. J. Mitchell, Nano Lett. 2021, 21, 5671.
[4] L. J. Kubiatowicz, A. Mohapatra, N. Krishnan, R. H. Fang, L. Zhang, Exploration 2022, 20210217.
[5] F. Ledesma, B. Ozcan, X. Sun, S. M. Medina, M. P. Landry, Adv. Funct. Mater. 2022, 32, 2107174.
[6] C. Webb, S. Ip, N. V. Bathula, P. Popova, S. K. V. Soriano, H. H. Ly, B. Eryilmaz, V. A. Nguyen Huu, R. Broadhead, M. Rabel, I. Villamagna, S. Abraham, V. Raeesi, A. Thomas, S. Clarke, E. C. Ramsay, Y. Perrie, A. K. Blakney, Mol. Pharm. 2022, 19, 1047.
[7] Y. Dong, D. J. Siegwart, D. G. Anderson, Adv. Drug Deliv. Rev. 2019, 144, 133.
[8] J. Li, Y. Wang, Y. Zhu, D. Oupicky, J. Control. Release 2013, 172, 589.
[9] J. B. Opalinska, A. M. Gewirtz, Nat. Rev. Drug Discov. 2002, 1, 503.
[10] Y.-K. Oh, T. G. Park, Adv. Drug Deliv. Rev. 2009, 61, 850.
[11] A. Da Silva Sanchez, K. Paunovska, A. Cristian, J. E. Dahlman, Hum. Gene Ther. 2020, 31, 940.
[12] H. J. Vaughan, J. J. Green, S. Y. Tzeng, Adv. Mater. 2020, 32, e1901081.
[13] J. A. Kulkarni, D. Witzigmann, S. B. Thomson, S. Chen, B. R. Leavitt, P. R. Cullis, R. van der Meel, Nat. Nanotechnol. 2021, 16, 630.
[14] L. Miao, Y. Zhang, L. Huang, Mol. Cancer 2021, 20, 41.
[15] N. Chaudhary, D. Weissman, K. A. Whitehead, Nat. Rev. Drug Discov. 2021, 20, 817.
[16] W. Ho, M. Gao, F. Li, Z. Li, X.-Q. Zhang, X. Xu, Adv. Healthc. Mater. 2021, 10, e2001812.
[17] Y. Yuan, Z. Gu, C. Yao, D. Luo, D. Yang, Small 2019, 15, e1900172.
[18] A. Gupta, J. L. Andresen, R. S. Manan, R. Langer, Adv. Drug Deliv. Rev. 2021, 178, 113834.
[19] E. Callaway, Nature 2020, 587, 337.
[20] L. Dai, G. F. Gao, Nat. Rev. Immunol. 2020, 21, 73.
[21] M. N. Uddin, M. A. Roni, Vaccines (Basel) 2021, 9, DOI 10.3390/vaccines9091033.
[22] K. S. Park, X. Sun, M. E. Aikins, J. J. Moon, Adv. Drug Deliv. Rev. 2021, 169, 137.
[23] J. L. Andresen, O. S. Fenton, MRS Bull. 2021, 46, 832.
[24] L. R. Baden, H. M. El Sahly, B. Essink, K. Kotloff, S. Frey, R. Novak, D. Diemert, S. A. Spector, N. Rouphael, C. B. Creech, J. McGettigan, S. Khetan, N. Segall, J. Solis, A. Brosz, C. Fierro, H. Schwartz, K. Neuzil, L. Corey, P. Gilbert, COVE Study Group, N. Engl. J. Med. 2021, 384, 403.
[25] E. E. Walsh, R. W. Frenck, A. R. Falsey, N. Kitchin, J. Absalon, A. Gurtman, S. Lockhart, K. Neuzil, M. J. Mulligan, R. Bailey, K. A. Swanson, P. Li, K. Koury, W. Kalina, D. Cooper, C. Fontes-Garfias, P.-Y. Shi, Ö. Türeci, K. R. Tompkins, K. E. Lyke, W. C. Gruber, N. Engl. J. Med. 2020, 383, 2439.
[26] M. Herrera, J. Kim, Y. Eygeris, A. Jozic, G. Sahay, Biomater. Sci. 2021, 9, 4289.
[27] I. M. S. Degors, C. Wang, Z. U. Rehman, I. S. Zuhorn, Acc. Chem. Res. 2019, 52, 1750.
[28] Y. J. Kwon, Acc. Chem. Res. 2012, 45, 1077.
[29] R. K. Singla, A. K. Dubey, A. Garg, R. K. Sharma, M. Fiorino, S. M. Ameen, M. A. Haddad, M. Al-Hiary, J. AOAC Int. 2019, 102, 1397.
[30] M. Leopoldini, N. Russo, M. Toscano, Food Chem. 2011, 125, 288.
[31] N. Kumar, N. Goel, Biotechnol Rep (Amst) 2019, 24, e00370.
[32] J. Chen, S. Pan, J. Zhou, Z. Lin, Y. Qu, A. Glab, Y. Han, J. J. Richardson, F. Caruso, Adv. Mater. 2022, 34, e2108624.
[33] M. Shin, J. H. Ryu, J. P. Park, K. Kim, J. W. Yang, H. Lee, Adv. Funct. Mater. 2015, 25, 1270.
[34] G. Yun, J. J. Richardson, M. Capelli, Y. Hu, Q. A. Besford, A. C. G. Weiss, H. Lee, I. S. Choi, B. C. Gibson, P. Reineck, F. Caruso, Adv. Funct. Mater. 2020, 30, 1905805.
[35] Y. Han, Z. Lin, J. Zhou, G. Yun, R. Guo, J. J. Richardson, F. Caruso, Angew. Chem. Int. Ed 2020, 59, 15618.
[36] J. Zhou, Z. Lin, Y. Ju, M. A. Rahim, J. J. Richardson, F. Caruso, Acc. Chem. Res. 2020, 53, 1269.
[37] D. Lin, B. Xing, Environ. Sci. Technol. 2008, 42, 5917.
[38] H. G. Nam, M. G. Nam, P. J. Yoo, J.-H. Kim, Soft Matter 2019, 15, 785.
[39] Y. Han, J. Zhou, Y. Hu, Z. Lin, Y. Ma, J. J. Richardson, F. Caruso, ACS Nano 2020, 14, 12972.
[40] J. J. Lu, R. Langer, J. Chen, Mol. Pharm. 2009, 6, 763.
[41] T. M. Allen, P. R. Cullis, Adv. Drug Deliv. Rev. 2013, 65, 36.
[42] B. L. Mui, Y. K. Tam, M. Jayaraman, S. M. Ansell, X. Du, Y. Y. C. Tam, P. J. Lin, S. Chen, J. K. Narayanannair, K. G. Rajeev, M. Manoharan, A. Akinc, M. A. Maier, P. Cullis, T. D. Madden, M. J. Hope, Mol. Ther. Nucleic Acids 2013, 2, e139.
[43] G. Sahay, W. Querbes, C. Alabi, A. Eltoukhy, S. Sarkar, C. Zurenko, E. Karagiannis, K. Love, D. Chen, R. Zoncu, Y. Buganim, A. Schroeder, R. Langer, D. G. Anderson, Nat. Biotechnol. 2013, 31, 653.
[44] I. S. Zuhorn, U. Bakowsky, E. Polushkin, W. H. Visser, M. C. A. Stuart, J. B. F. N. Engberts, D. Hoekstra, Mol. Ther. 2005, 11, 801.
[45] F. M. van de Water, O. C. Boerman, A. C. Wouterse, J. G. P. Peters, F. G. M. Russel, R. Masereeuw, Drug Metab. Dispos. 2006, 34, 1393.
[46] T. Bondue, L. van den Heuvel, E. Levtchenko, R. Brock, Pediatr. Nephrol. 2022, DOI 10.1007/s00467-021-05352-w.
[47] A. P. French, S. Mills, R. Swarup, M. J. Bennett, T. P. Pridmore, Nat. Protoc. 2008, 3, 619.
[48] L. I. Selby, C. M. Cortez-Jugo, G. K. Such, A. P. R. Johnston, Wiley Interdiscip. Rev. Nanomed. Nanobiotechnol. 2017, 9, DOI 10.1002/wnan. 1452.

### Example 2. A Mechanistic Evaluation of Polyphenol Incorporated Lipid Nanoparticles for mRNA Delivery

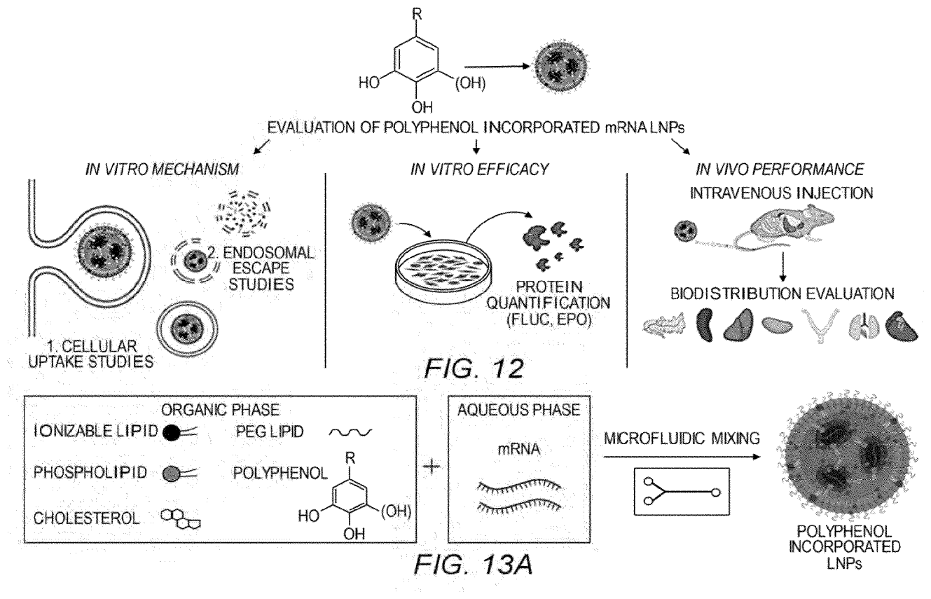

Expanding upon the research, the objective of this study is to create a library of polyphenol incorporated LNPs and compare their key physio-chemical properties, cellular performance, as well as their in vitro and in vivo efficacy. To achieve this goal, polyphenol based LNPs were synthesized utilizing varying molecular weight (Mw) polyphenols, including gallic acid (GA), catechin (CAT), epigallocatechin gallate (EGCG) and TA. First, the key physio-chemical properties of these LNPs were evaluated, such as size, polydispersity index (PDI), zeta potential, encapsulation efficiency, stability, and the predominant driving force behind LNP formation and stability. Furthermore, comprehensive evaluation of the cellular performance was performed, including endocytosis mechanisms, cellular uptake dynamics over various time intervals, endosomal escape processes and their underlying mechanisms on B16-F10 melanoma tumor and DC 2.4 dendritic cells (DCs) since LNPs can be potentially used in the field of immunotherapy. Finally, in vitro efficacy was assessed by using mRNA encoded protein expression of both intracellular (e.g., FLuc) and secreted proteins (e.g., EPO) at various doses and times (FIG. 12). Notably, the findings reveal that LNPs formulated with high Mw polyphenols, such as EGCG LNPs and TA LNPs, exhibited superior in vivo FLuc and EPO expression (?) compared to LNPs alone, despite contrasting in vitro results. Of note, the analysis of blood paneling, histological assessments, and weight loss data indicate that polyphenol mediated LNPs are all well tolerated in vivo, bearing no statistical differences to the tolerability profiles observed in mice treated with a PBS control group. In conclusion, this work shows that the in vitro performance of LNPs formulated with polyphenols of varying Mw may not necessarily correlate with their in vivo efficacy.

### Synthesis and Characterization of Polyphenol Incorporated LNPs

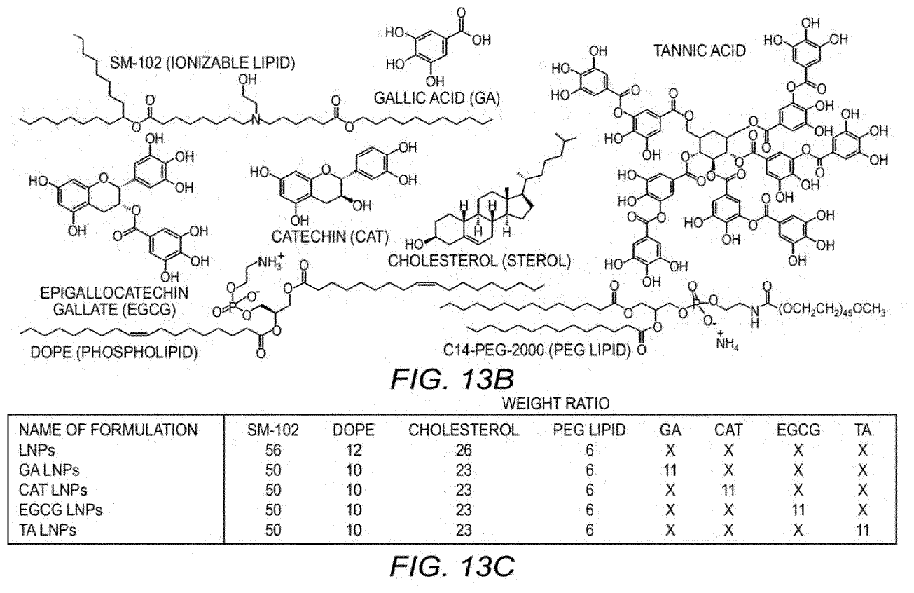

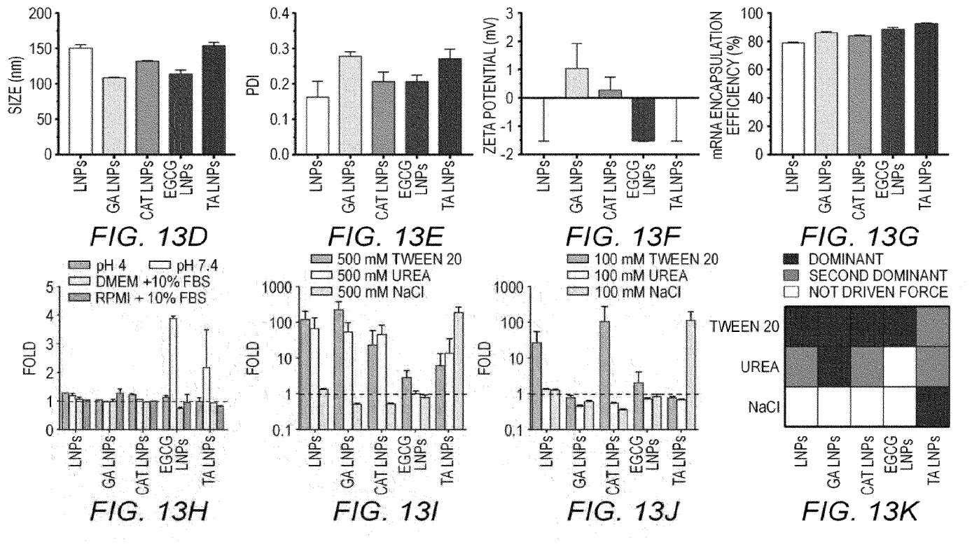

Toward the goal of investigating the effect of polyphenols on the activity of LNPs, LNPs were formulated with various molecular weight (Mw) polyphenols noted as GA LNPs, CAT LNPs, EGCG LNPs and TA LNPs. Briefly, LNPs were formulated via microfluidic chip by mixing an aqueous phase containing mRNA with an ethanol phase containing different types of ionizable lipids (either Moderna's SM-102 or Pfizer/BioNTech's ALC-0315, to maximize translational relevance),^{[1.2]} the phospholipid DOPE (which was used because it was previously observed to improve the efficacy of LNPs relative to DSPC),^{[3-5]} cholesterol,^{[6,7]} lipid anchored polyethylene glycol, and a polyphenol (GA, CAT, EGCG or TA)^{[4,8,9]} (FIGS. 13A-13C). The size, charge, and encapsulation efficiency for each LNP was reproducible, with sizes ranging from ˜109 nm to ˜154 nm (FIG. 13D), PDI ranging from ˜0.16 to ˜0.28 (FIG. 13E), zeta potentials ranging from ˜-1.5 mV to ˜1.0 mV (FIG. 13F), and mRNA encapsulation efficiencies of LNPs ranging from ˜78.9% to ˜92.4% (FIG. 13G). To begin to study the role of polyphenols on LNPs their relative stability in physiological conditions was first studied. It was found each LNP formulation was stable under different conditions (pH 4, pH 7.4, DMEM+10% FBS and RPMI+10% FBS) at 37° C. for 48 h, except EGCG LNPs and TA LNPs at pH 7.4 buffer as indicated by the increase in size. (FIG. 13H). To further investigate the reason behind the differences in stability, the molecular interactions which contribute to LNP formation were studied, in which each LNP formulation was incubated with urea (disrupts hydrogen bonding), tween 20 (disrupts hydrophobic interactions) and NaCl (disrupts electrostatic interactions) (FIGS. 13I-13K). Several trends were observed through studying the underlying molecular interactions of each LNP formulation. First, it was shown electrostatic interactions were the main driving force in the formation of TA LNPs but not for other formulations as indicated by the size increase in TA LNPs (highest Mw polyphenol) when incubated with 500 mM NaCl.

Additionally, both hydrogen bonding and hydrophobic interactions were the main driving force for each LNP formulation as indicated by size increases in 500 mM urea and 500 mM tween 20.

### Assessment of Cellular Uptake and Degradation of Polyphenol Mediated LNPs

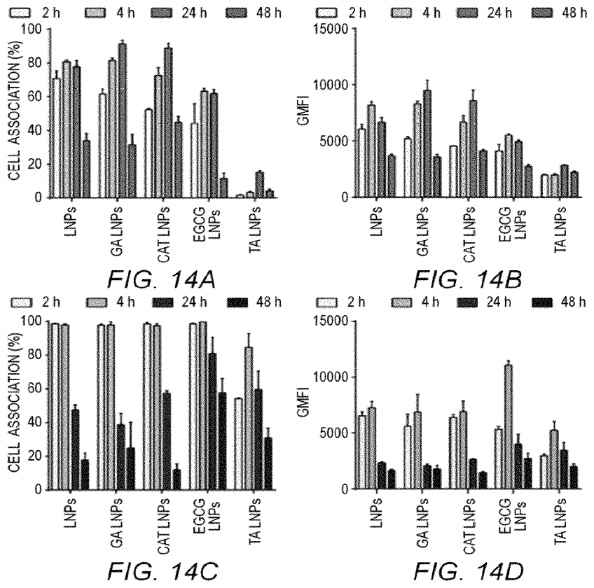

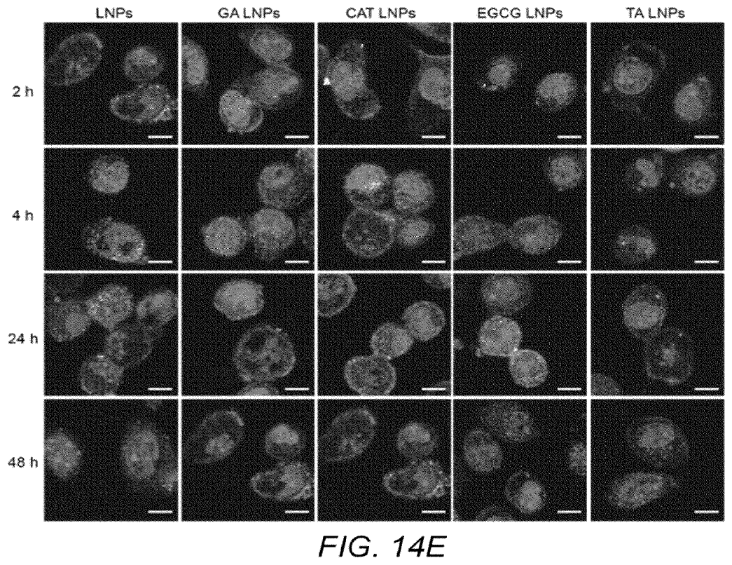

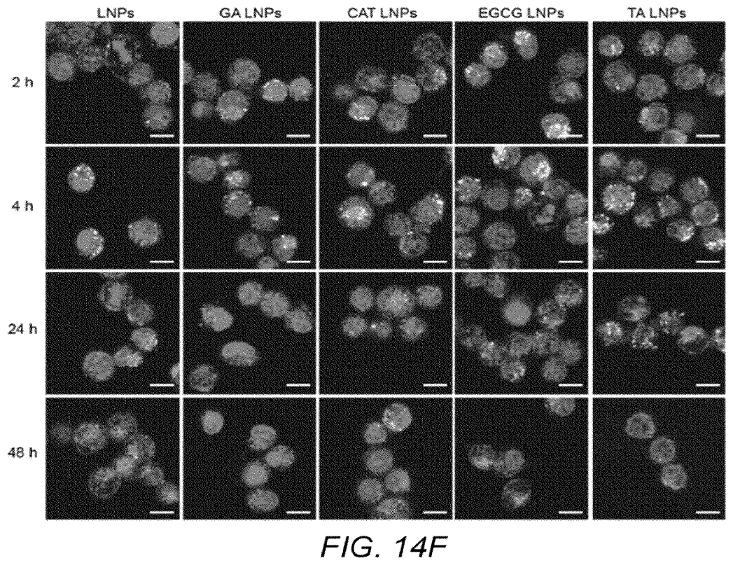

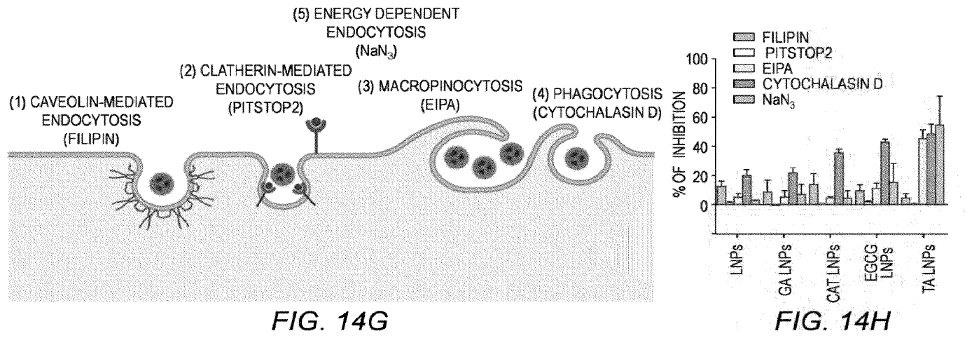

Following the characterization of the LNPs, several key mechanisms thought to contribute to polyphenol mediated LNP delivery and efficacy were studied. To begin, the internalization efficiency of the LNPs were studied in vitro. Internalization efficiency was defined by the percent of cells which have taken up LNP, and was quantified using flow cytometry and confocal microscopy. The internalization efficiency of each LNP formulation was studied as a function of incubation time (2, 4, 24, and 48 h) using ATTO-488 labelled LNPs on B16-F10 cells (melanoma cell) and DC 2.4 cells (dendritic cells) due to the potential vaccine application of mRNA LNPs (FIGS. 14A-14F, Table 3). To couple with the internalization efficiency, elucidation of the specific cellular uptake pathway and degradation properties of each polyphenol LNP formulation in vitro was sought. Through inhibition of various endocytic pathways, specifically caveolin-mediated endocytosis, clathrin-dependent endocytosis, micropinocytosis, phagocytosis, and energy-dependent endocytosis, the mechanism behind polyphenol LNP cell uptake was elucidated. Several trends emerged upon analyzing these studies. First, TA LNPs had lower cellular uptake compared to other LNP formulations at various time periods (FIG. 14A, 14C). Second, the cellular internalization of LNPs was time dependent, and maximum uptake in each LNP formulation was seen at 24 h for B16-F10 cells and 4 h for DC 2.4 cells (FIG. 14A, 14C). Third, each LNP formulation degraded after 24 h in B16-F10 cells and 4 h in DC 2.4 cells (FIG. 14A, 14C) indicated through the decrease in geometric mean fluorescence intensity (GMFI) after 24 h for B16-F10 cells and 4 h for DC 2.4 cells (FIG. 14B, 14D), additionally shown in the confocal microscopy images (FIG. 14E, 14F). And finally, phagocytosis was shown to be the main mechanisms for non-TA LNP uptake, while micropinocytosis and energy-dependent endocytosis were the two main mechanisms for TA LNPs, possibly due to electrostatic interactions being the primary driving force to formulate TA LNPs (FIG. 14G, 14H). Taken in tandem, these results suggest that LNPs are internalized in a time dependent fashion through a combination of endocytic pathways (24 h on B16-F10 cells and 4 h on DC 2.4 cells), and as a result begin to degrade upon internalization. (after 24 h on B16-F10 cells and 4 h on DC 2.4 cells).

### Endosomal Escape of Polyphenol Mediated LNPs

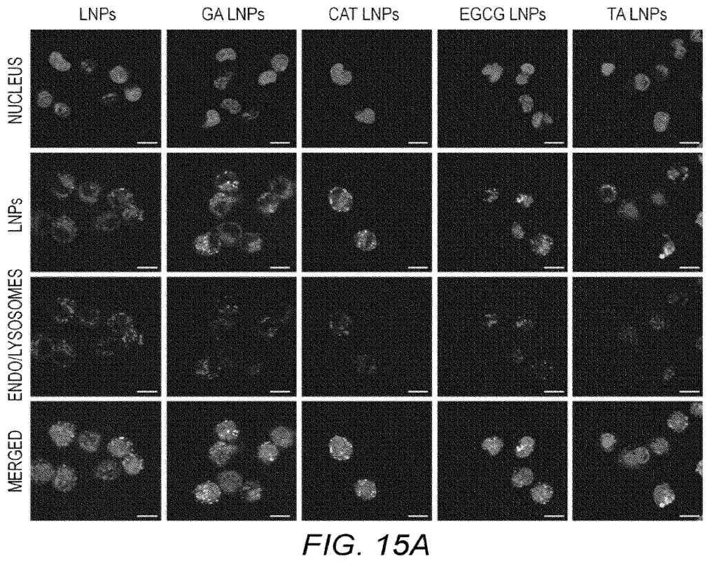

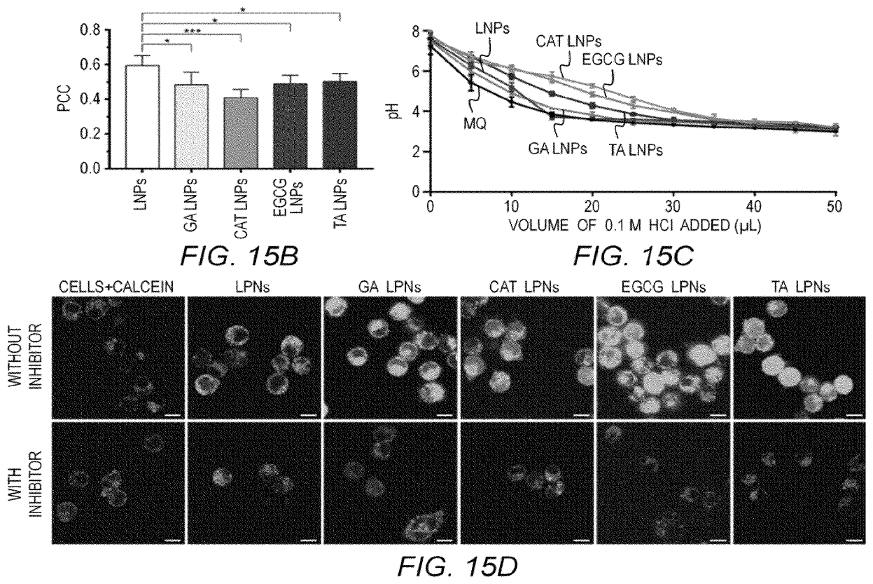

Following cellular internalization/uptake studies, an understanding of the potential differences in endosomal escape of the polyphenol mediated LNPs was sought as it has been hypothesized endosomal escape might contribute to overall efficacy of mRNA LNPs. These endosomal escape studies were investigated by incubating DC 2.4 cells with ATTO-488 labelled FLuc mRNA polyphenol LNPs and imaging endosomal escape using confocal microscopy. To quantify the level of escape the Pearson Coefficient Correlation (PCC) was used, where a value of 0 indicates complete endosomal escape and a value of 1 indicates no endosomal escape^{[10]} (FIG. 15A, 15B). In these confocal images, cell nuclei are medium gray, mRNA LNPs are gray, endo/lysosomes are dark gray, and mRNA LNPs trapped in endosomes are light gray (i.e., colocalization of the gray and dark gray signals). To provide further structural and physiochemical insights into the polyphenol LNPs the specific molecular mechanism of polyphenol mediated LNPs which contributes to endosomal escape through a buffering capacity study was investigated. In this study, HCl was gradually added to each LNP formulation and the pH of the LNP was measured at various HCl volumes (FIG. 15C). As a means to study the biological mechanisms with contribute to the endosomal escape of polyphenol mediated LNPs, enzyme inhibition/brightfield imaging studies with bafilomycin A₁ (an inhibitor of V-ATPases whose inhibition terminates endosomal and lysosomal acidification processes that can impact the endosomal escape of LNPs)^{[11,12]} and calcein (a membrane-impermeable dye that remains entrapped within intact endosomes but becomes distributed throughout cells if endo/lysosomes are ruptured) were performed (FIG. 15D).

Upon analyzing this data, several findings were observed. First, polyphenol mediated LNPs had better endosomal escape than LNPs due to their significantly lower PCC value (FIG. 15B). Second, the pH of high Mw polyphenol LNPs (CAT, EGCG and TA) more gradually decreased as compared to the low Mw polyphenol LNPs (GA), LNPs and MQ (as control), which indicates polyphenol LNPs formulated with high Mw polyphenols can buffer enough proton inflow to trigger endosomal escape (FIG. 15C). Third, diffuse fluorescence was observed when cells were incubated with polyphenol mediated LNPs compared to LNPs (upper row, FIG. 15D) further indicating the better endosomal escape of polyphenol mediated LNPs. Fourth, interspersed fluorescence was observed with cells treated with bafilomycin A₁ and polyphenol mediated LNPs (inhibiting influx of H⁺ and Cl⁻ into the endo/lysosomal membrane) (bottom row, FIG. 15D), suggesting that the ‘proton sponge’ could potentially be one of the mechanisms for triggering endosomal escape of polyphenol mediated LNPs. Taken in tandem, these results suggest that physiochemical properties of polyphenol mediated LNPs may impact endosomal escape through buffering of endosomal acidification and triggering ‘proton sponge’ effects.

### In Vitro Transfection of Polyphenol Mediated LNPs

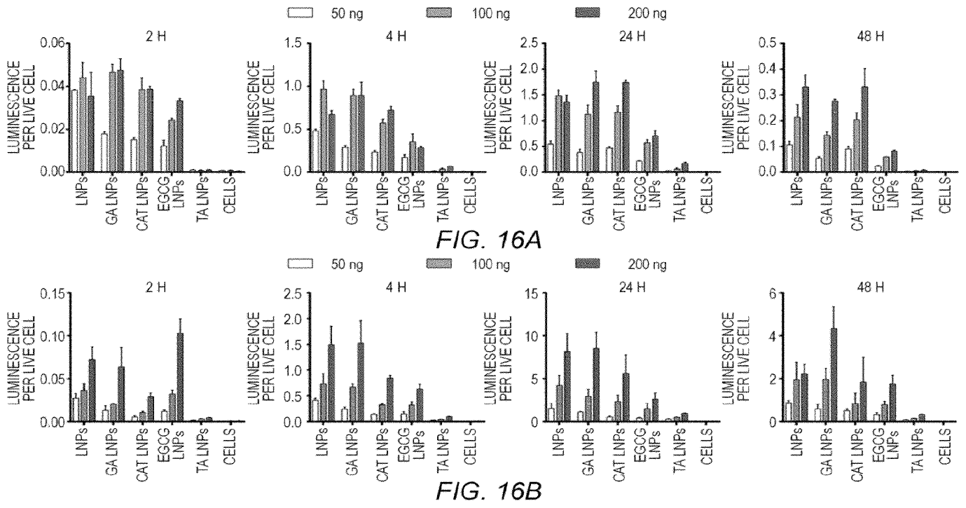

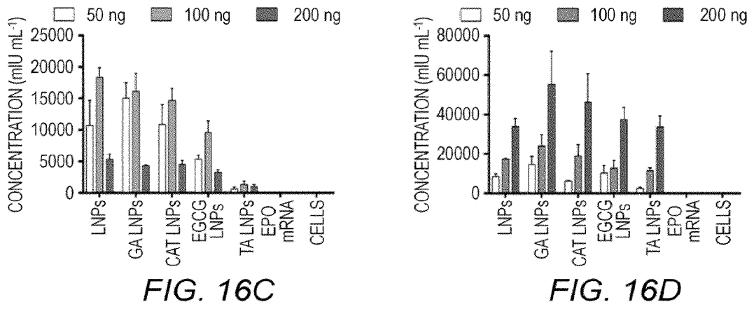

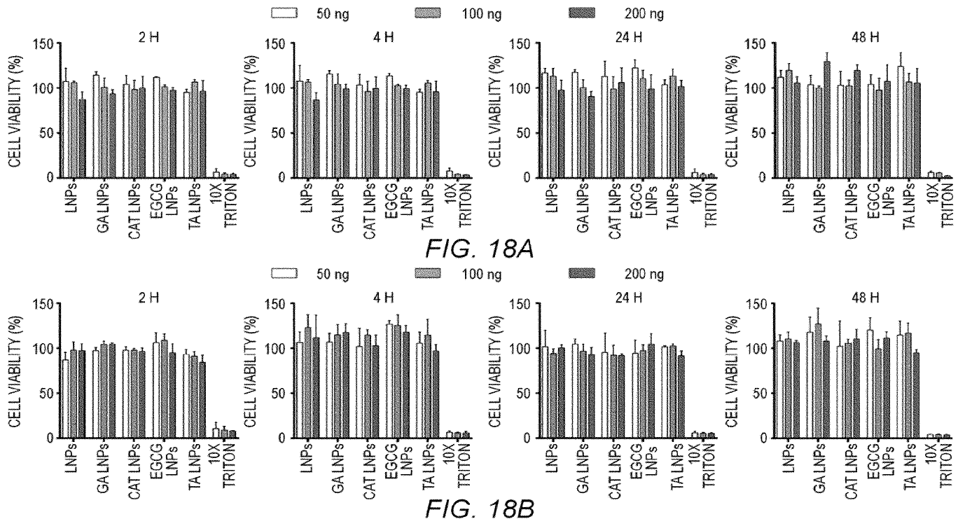

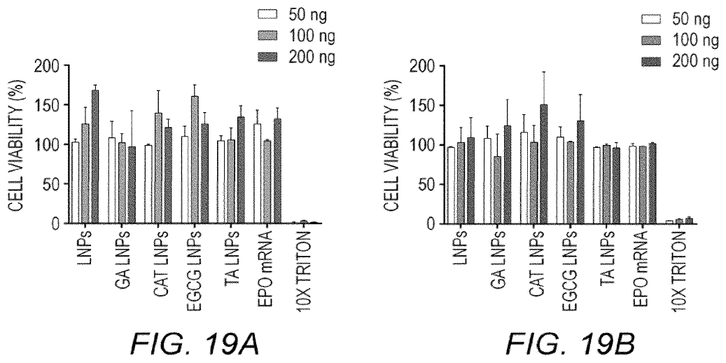

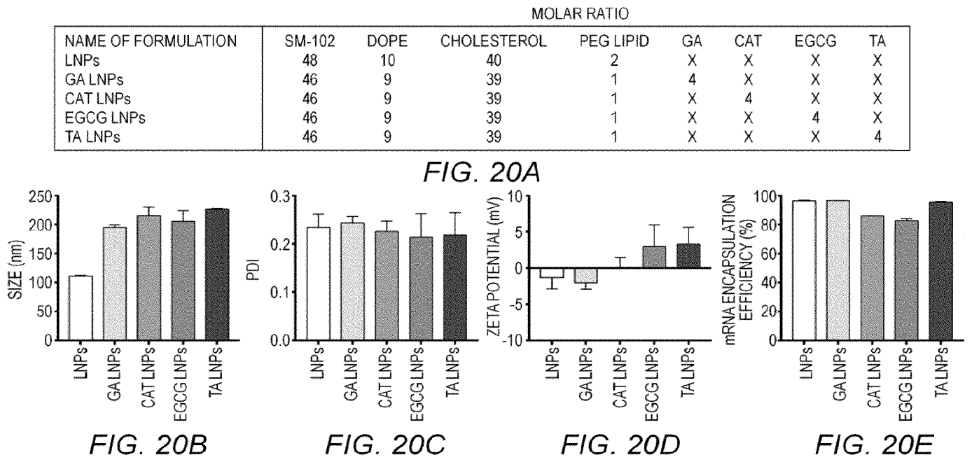

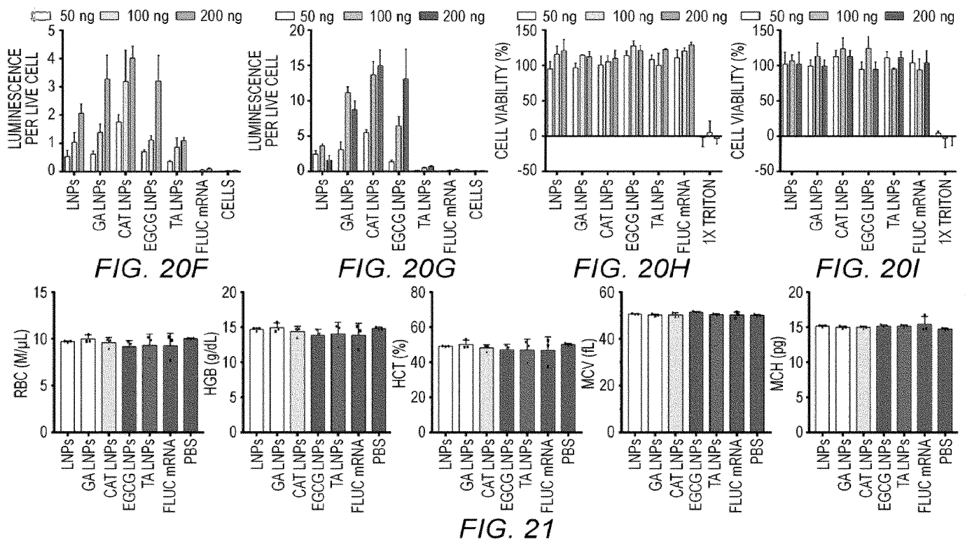

Upon studying the physiochemical properties of the polyphenol LNPs, the in vitro efficacy of polyphenol mediated LNPs was investigated by evaluating the protein expression in a dose response (50 ng, 100 ng and 200 ng) and a time dependent (2 h, 4 h, 24 h and 48 h) fashion using mRNA encoding for either firefly luciferase (FLuc, an intracellular protein) or human erythropoietin (EPO, a secreted protein) (FIG. 16, Table 4). Upon analyzing this data, several trends emerged. First, polyphenol mediated LNPs were well-tolerated under each studied condition (FIGS. 18, 19). Second, FLuc and EPO expressions for each LNP formulation were higher on DC 2.4 cells than B16-F10 cells. Third, FLuc expression for each LNP formulation increased from 2 h to 24 h followed by a decrease in expression following 24 h. (FIG. 16A, 16B). Fourth, FLuc expression at 24 hours responded in a dose responsive fashion for each LNP formulation as indicated by the increased expression correlating to increase in dose ranging from 50 ng to 200 ng. (FIG. 16A, 16B). Alternatively, treatment of LNPs encapsulating mRNA encoding for EPO on B16-F10 cells and DC 2.4 cells after 24 h showed highest EPO expression at 100 ng and 200 ng respectively. Finally, it was seen the higher Mw polyphenol formulated LNPs correlated to lower in vitro protein expression across both cell lines, possibly corresponding to the low cellular uptake as evidenced by TA LNP shown in FIG. 14. Notably, CAT LNPs (medium Mw polyphenol) exhibited the highest FLuc expression, when polyphenol LNPs were formulated at the same molar ratio (FIG. 20).

### In Vivo Transfection of Polyphenol Mediated LNPs

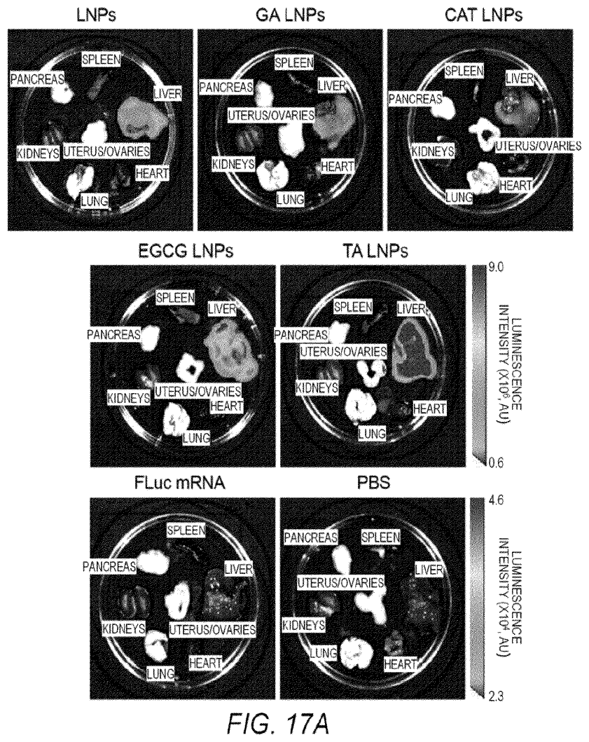

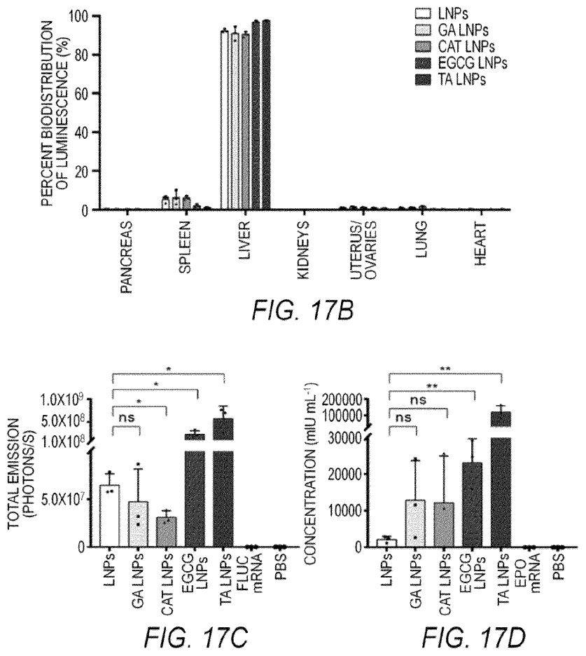

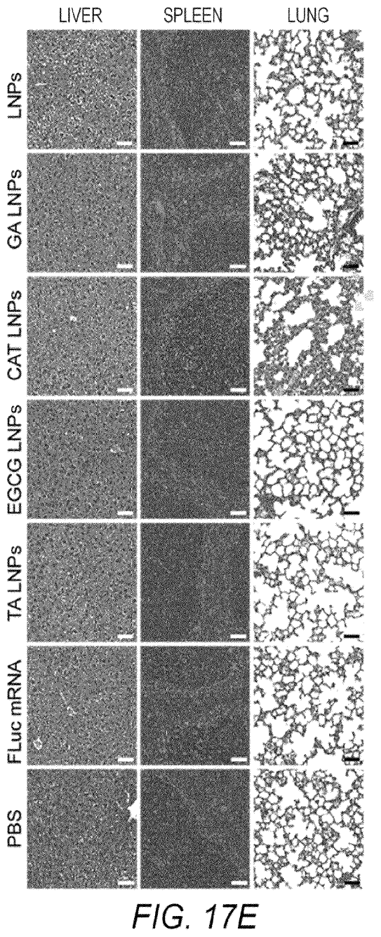

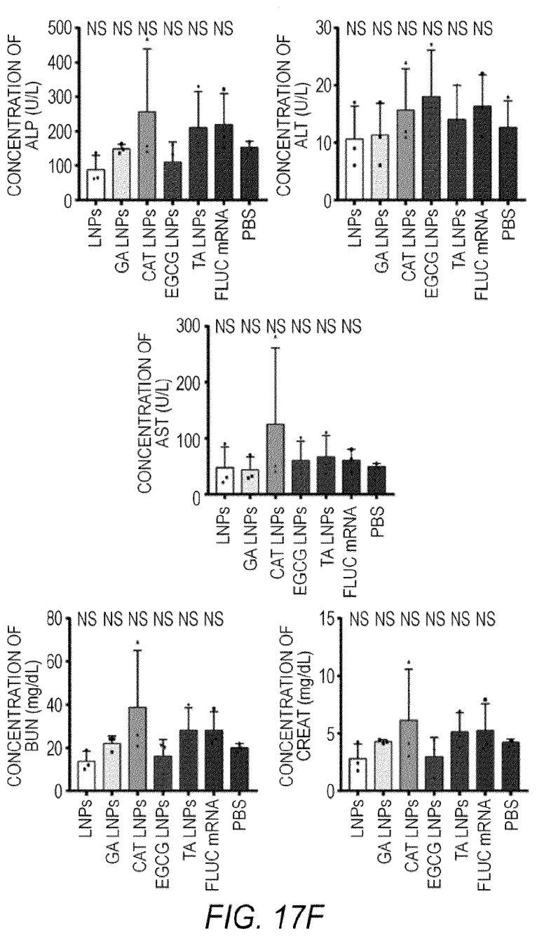

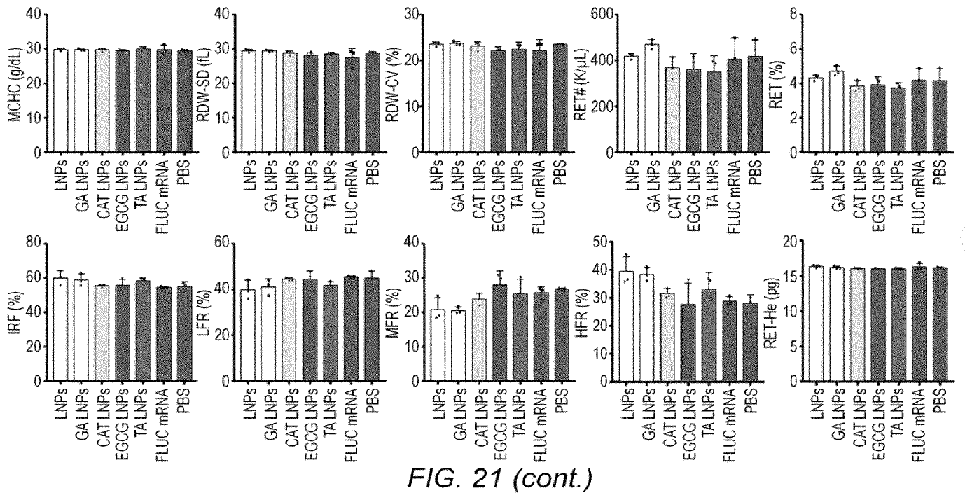

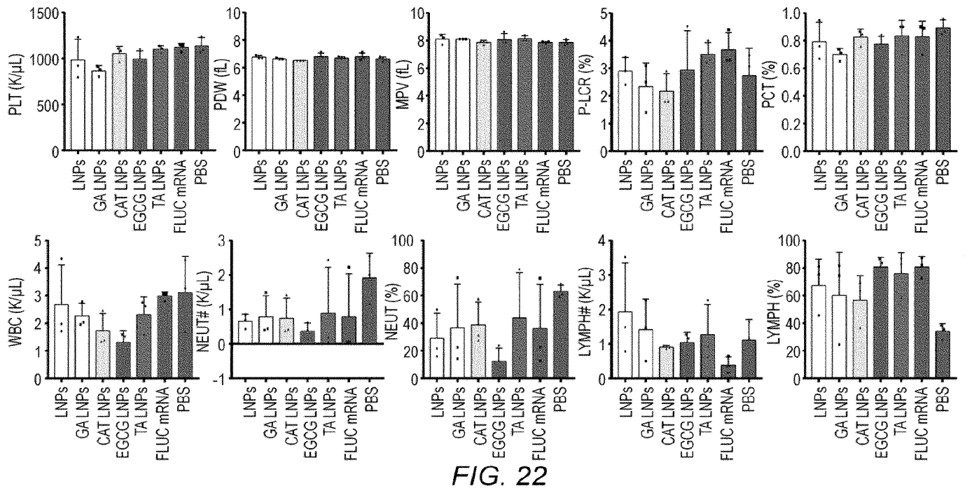

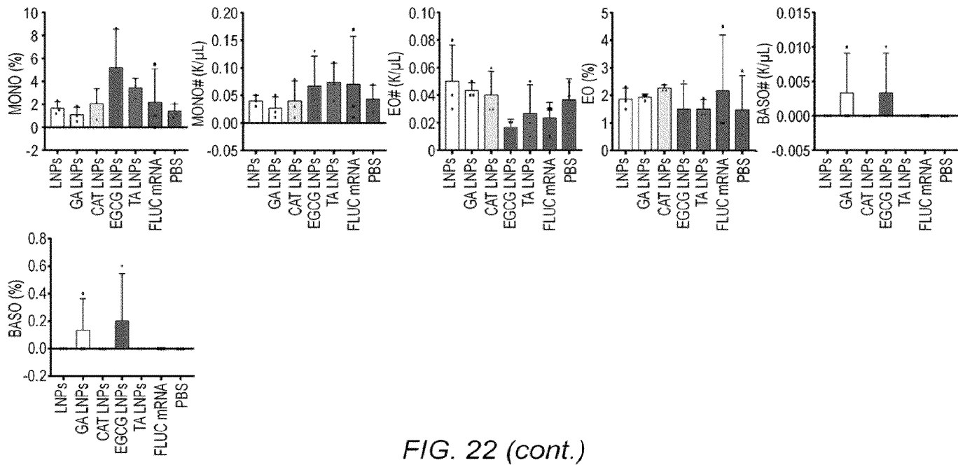

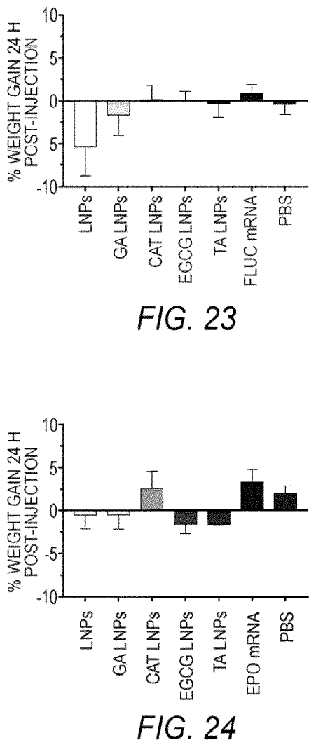

Building on the previous data, in vivo FLuc and EPO expression of polyphenol mediated LNPs was investigated. Briefly, Black 6 mice were treated with each LNP formulation delivering mRNA encoding for FLuc (FIGS. 17A-17C, Table 5) or EPO (FIG. 17D, Table 6) via intravenous (IV) administration. Tolerability studies including histological evaluation (FIG. 17E), liver and kidney function blood tests (FIG. 17F), complete blood paneling (FIGS. 21, 22), and weight loss studies (FIGS. 23, 24) were also evaluated for each LNP formulation. Upon analyzing this data, several trends were observed. First, polyphenol mediated LNPs with high Mw such as EGCG LNPs and TA LNPs have higher FLuc expression than LNPs as evidenced by the increased FLuc signal in comparative IVIS imaging on the resected organs of treated mice (FIG. 17A). Second, significantly higher FLuc signal was observed in mice treated with high Mw polyphenol LNPs (EGCG LNPs and TA LNPs) vs LNPs (FIG. 17C). Third, this increase in FLuc expression occurred without altering the innate biodistribution of each studied LNP formulation (FIG. 17B). Fourth, high Mw polyphenol LNPs (EGCG LNPs and TA LNPs) also increased the amount of EPO expression secreted into the blood of treated mice (FIG. 17D). Fifth, each LNP formulation was well tolerated as analyzed by histology (FIG. 17E), weight retention (FIGS. 23, 24), and complete blood paneling data including normal alkaline phosphatase (ALP), alanine transaminase (ALT), aspartate transferase (AST), blood urea nitrogen (BUN) and creatinine (CREAT) levels which are markers of liver and kidney function (FIG. 17F). Taken in tandem, these results suggest that high Mw polyphenol LNPs such as EGCG LNPs and TA LNPs had better in vivo performance than LNPs itself, but were opposite in their in vitro performance.

### Conclusion

In this report, a novel category of polyphenol-mediated LNPs was successfully developed, demonstrating their effectiveness as mRNA delivery systems by evaluating their physiochemical properties including the size, PDI, charge, encapsulation efficiency, stability, predominant driving force behind LNP formulation, and the mechanisms behind their cellular performance such as endocytosis and endosomal escape mechanisms. Furthermore, the research also showed that TA LNPs, formulating with highest Mw polyphenol, exhibited highest FLuc and EPO protein expression, but opposite in their in vitro performance in which the lowest FLuc and EPO protein expression was observed. This underscores the multifaceted nature of drug delivery systems, where the behavior and performance of LNPs in cell culture settings may not always accurately predict their behavior within the complex and dynamic environment of living organisms. This finding further suggests that the importance of conducting comprehensive in vivo studies to gain a more complete understanding of the true therapeutic potential of polyphenol-incorporated LNPs, offering valuable insights into their application as drug delivery platforms for future gene therapies and vaccines.

TABLE 3
Size, zeta, EE, PDI of animal ATTO-
488 labelled LNPs-FLuc mRNA in vitro

| | | | | FLuc mRNA |
|---|---|---|---|---|
| | | | | Encap- |
| | | | Zeta | sulation |
| | | | potential | Efficiency |
| | Size (nm) | PDI | (mV) | (%) |
| LNPs | 147.1 ± 4.2 | 0.25 ± 0.02 | −2.30 ± 2.66 | 77.7 ± 0.1 |
| GA LNPs | 102.3 ± 2.0 | 0.14 ± 0.05 | 0.39 ± 1.44 | 82.9 ± 0.2 |
| CAT LNPs | 124.4 ± 1.5 | 0.17 ± 0.06 | 0.20 ± 0.41 | 74.7 ± 0.8 |
| EGCG LNPs | 128.8 ± 1.3 | 0.22 ± 0.04 | −0.27 ± 1.51 | 88.0 ± 0.2 |
| TA LNPs | 153.2 ± 3.1 | 0.25 ± 0.03 | 2.59 ± 4.21 | 81.1 ± 0.4 |

TABLE 4
Size, zeta, EE, PDI of LNPs-EPO mRNA in vitro

| | | | | EPO |
|---|---|---|---|---|
| | | | | mRNA |
| | | | | Encap- |
| | | | Zeta | sulation |
| | | | potential | Efficiency |
| | Size (nm) | PDI | (mV) | (%) |
| LNPs | 172.8 ± 4.0 | 0.23 ± 0.08 | −2.38 ± 5.30 | 82.2 ± 1.7 |
| GA LNPs | 195.6 ± 8.8 | 0.29 ± 0.02 | 2.00 ± 3.77 | 73.7 ± 1.4 |
| CAT LNPs | 162.3 ± 4.4 | 0.29 ± 0.01 | 0.69 ± 1.96 | 88.1 ± 1.0 |
| EGCG LNPs | 189.5 ± 17.2 | 0.25 ± 0.07 | 2.31 ± 5.43 | 90.4 ± 1.9 |
| TA LNPs | 203.6 ± 6.0 | 0.30 ± 0.02 | −1.87 ± 4.58 | 85.8 ± 1.6 |

TABLE 5
Size, zeta, EE, PDI of animal injected LNPs-FLuc mRNA in vivo

| | | | | FLuc mRNA |
|---|---|---|---|---|
| | | | | Encap- |
| | | | Zeta | sulation |
| | | | potential | Efficiency |
| | Size (nm) | PDI | (mV) | (%) |
| LNPs | 147.2 ± 4.1 | 0.27 ± 0.01 | 1.91 ± 2.44 | 85.3 ± 0.1 |
| GA LNPs | 133.2 ± 4.1 | 0.26 ± 0.03 | 1.71 ± 3.26 | 90.1 ± 3.2 |
| CAT LNPs | 133.2 ± 5.4 | 0.24 ± 0.06 | −0.78 ± 2.94 | 89.0 ± 0.4 |
| EGCG LNPs | 142.7 ± 1.0 | 0.18 ± 0.01 | −1.33 ± 0.75 | 92.0 ± 0.3 |
| TA LNPs | 162.8 ± 2.9 | 0.28 ± 0.01 | 1.88 ± 1.89 | 85.3 ± 0.6 |

TABLE 6
Size, zeta, EE, PDI of animal injected LNPs-EPO mRNA in vivo

| | | | | EPO mRNA |
|---|---|---|---|---|
| | | | | Encap- |
| | | | Zeta | sulation |
| | | | potential | Efficiency |
| | Size (nm) | PDI | (mV) | (%) |
| LNPs | 126.1 ± 0.5 | 0.26 ± 0.02 | 0.03 ± 1.53 | 88.9 ± 0.9 |
| GA LNPs | 134.3 ± 1.5 | 0.28 ± 0.02 | 5.48 ± 5.77 | 87.3 ± 0.3 |
| CAT LNPs | 130.5 ± 11.9 | 0.26 ± 0.03 | 1.60 ± 3.11 | 85.2 ± 0.1 |
| EGCG LNPs | 132.8 ± 1.7 | 0.25 ± 0.01 | 1.98 ± 2.24 | 90.8 ± 0.6 |
| TA LNPs | 181.1 ± 3.7 | 0.21 ± 0.04 | 4.77 ± 6.01 | 92.2 ± 0.7 |

### REFERENCES

[1] L. Schoenmaker, D. Witzigmann, J. A. Kulkarni, R. Verbeke, G. Kersten, W. Jiskoot, D. J. A. Crommelin, Int. J. Pharm. 2021, 601, 120586.
[2] R. Tenchov, R. Bird, A. E. Curtze, Q. Zhou, ACS Nano 2021, 15, 16982.
[3] I. S. Zuhorn, U. Bakowsky, E. Polushkin, W. H. Visser, M. C. A. Stuart, J. B. F. N. Engberts, D. Hoekstra, Mol. Ther. 2005, 11, 801.
[4] Y. Ma, O. S. Fenton, Adv. Therap. 2023, 2200305.
[5] Y. Ma, O. S. Fenton, J. Am. Chem. Soc. 2023, DOI 10.1021/jacs.3c02584.
[6] J. J. Lu, R. Langer, J. Chen, Mol. Pharm. 2009, 6, 763.
[7] T. M. Allen, P. R. Cullis, Adv. Drug Deliv. Rev. 2013, 65, 36.
[8] Y. Han, J. Zhou, Y. Hu, Z. Lin, Y. Ma, J. J. Richardson, F. Caruso, ACS Nano 2020, 14, 12972.
[9] Y. Qu, Y. Ju, C. Cortez-Jugo, Z. Lin, S. Li, J. Zhou, Y. Ma, A. Glab, S. J. Kent, F. Cavalieri, F. Caruso, Small 2020, 16, e2002750.
[10] A. P. French, S. Mills, R. Swarup, M. J. Bennett, T. P. Pridmore, Nat. Protoc. 2008, 3, 619.
[11] Y. Hu, T. Litwin, A. R. Nagaraja, B. Kwong, J. Katz, N. Watson, D. J. Irvine, Nano Lett. 2007, 7, 3056.
[12] S. Drose, K. Altendorf, J. Exp. Biol. 1997, 200, 1.

### Experimental Section

### Materials

Phenazine methosulfate (PMS), tannic acid (TA), Dulbecco's phosphate-buffered saline (DPBS), calcium chloride, sodium phosphate dibasic, phosphate-buffered saline (PBS, pH 7.4), cholesterol, sodium citrate, citrate acid, penicillin-streptomycin, Triton X-100, NaN3, filipin from S. filipinensis, cytochalasin D, (N-ethyl-N-isopropyl) amiloride (EIPA), ATTO-488 DOPE, tween 20, urea, and sodium chloride were purchased from Sigma-Aldrich. 1,2-dioleoyl-sn-glycero-3-phosphoethanolamine (DOPE) and 1,2-dimyristoyl-sn-glycero-3-phosphoethanolamine-N-[methoxy-(polyethyleneglycol)-2000] (ammonium salt) (C14-PEG 2000) were purchased from Avanti. Moderna SM-012 Lipids was bought from Broadpharm. Dulbecco's modified Eagle's medium (DMEM), RPMI 1640, Fetal Bovine Serum (FBS), calcein, LysoTracker Deep Red, 2,3-Bis [2-methoxy-4-nitro-5-sulfophenyl]-2H-tetrazolium-5-carboxyanilide inner salt (XTT), trihydrochloride (Hoechst 33342, 10 mg mL⁻¹) were purchased from Life Technologies. Firefly luciferase mRNA, Human EPO mRNA and OVA mRNA were obtained from TriLink Biotechnologies. (−)-Epigallocatechin gallate (EGCG), gallic acid monohydrate (GA), (+)-catechin hydrate (CAT), Quant-iT RiboGreen RNA assay kit, bafilomycin A₁, tumor necrosis factor (TNF)-α mouse ELISA kit, and Human EPO ELISA kit were purchased from Thermo fisher Scientific. Bright-Glo Luciferase assay system was obtained from Promega. Pitstop 2 was purchased from Abcam. Ultrapure water (Milli-Q) with a resistivity greater than 18.2 MQ2 cm was used in all experiments and obtained from a three stage Millipore Milli-Q Plus 185 purification system. All chemicals were used without further purification.

### Synthesis of Lipid Nanoparticles

Ethanol phase was prepared by mixing Moderna SM-102 ionizable lipid, dioleoyl-sn-glycero-3-phosphoethanolamine, cholesterol, 1,2-dimyristoyl-sn-glycero-3 phosphoethanolamine-N-[methoxy-(polyethyleneglycol)-2000] (ammonium salt) (C14-PEG 2000) with or without various polyphenols including GA, CAT, EGCG and TA. Aqueous phase was made by dissolving the 0.1 mg mL⁻¹ of FLuc/EPO mRNA in 10 mM citrate buffer (pH 3). Then, the aqueous and ethanol phases were mixed at a ratio of 3:1 into a microfluid chip device. Finally, the synthesized LNPs were dialyzed against 1×PBS in a 20,000 MWCO cassette at 4° C. cold room for 2 h, followed by storing at 4° C. prior for further use.

### Characterization

Size distribution, zeta potentials of LNPs, LNPs with various polyphenols such as GA LNPs, CAT LNPs, EGCG LNPs, TA LNPs were measured using a dynamic light scattering (DLS) measurement (NanoBrook 90 Plus Zeta, Brookhaven, USA). The sample were diluted in 1×PBS (pH 7.4) for the size measurement and 0.1×PBS (pH 7.4) for zeta potential measurement. The FLuc/EPO mRNA encapsulation efficiency was quantified by Quant-iT RiboGreen RNA assay kit a SpectraMax microplate reader (Molecular Device, USA) with 480 nm excitation wavelength and 520 nm emission wavelength. To characterize the overall mRNA concentration, the LNPs were fully dissolved with 1× triton.

### Stability of Lipid Nanoparticles

FLuc mRNA encapsulated LNPs, GA LNPs, CAT LNPs, EGCG LNPs, and TA LNPs were incubated with 1×pH 4, pH 7.4 buffer, DMEM+10% FBS and RPMI+10% FBS media at 37° C. for 48 h. The size was determined by NanoBrook 90 Plus Zeta via diluting the samples in 1×PBS (pH 7.4) buffer. The ratio of the size of LNPs after 48 h vs, the size at 0 h was plotted to indicate the stability of the LNPs.

### Predominant Interaction Formulation of Assembled Lipid Nanoparticles

20 μL of FLuc mRNA encapsulated LNPs, GA LNPs, CAT LNPs, EGCG LNPs, and TA LNPs suspensions were dissolved in 100 μL of either 500 mM or 100 mM urea, 100 mM Tween 20, or 100 mM NaCl solution and incubated in an Eppendorf thermomixer at 37° C. and 500 rpm for 48 h. The suspensions were further diluted with 1×PBS (pH 7.4) buffer and the size changes of LNPs were measured via dynamic light scattering (DLS) measurements. The ratio of the size of LNPs after 48 h vs, the size at 0 h was plotted to indicate the predominate interactions of the formulation of LNPs.

### In Vitro Dose Response of FLuc Expression

B16-F10 and DC 2.4 cells were seeded at a density of 1×10⁴ cells per well in 96 well plates at 37° C. Next day, the cell media was aspirated and 100 μL of 500 ng mL⁻¹, 1000 ng mL⁻¹ or 2000 ng mL⁻¹ of FLuc mRNA encapsulated LNPs, GA LNPs, CAT LNPs, EGCG LNPs, TA LNPs, and naked FLuc mRNA were added into the seeded B16-F10 and DC 2.4 cells followed by incubation for various time (2, 4, 24 and 48 h). Subsequently, 100 μL of Bright-Glo Luciferase assay buffer was added into each treated well, and the transfection value was measured using a SpectraMax microplate reader with luminescence function.

### In Vitro Dose Response of EPO Expression

B16-F10 and DC 2.4 cells were seeded at a density of 1×10⁴ cells per well in 96 well plates at 37° C. Next day, the cell media was aspirated and 100 μL of 500 ng mL⁻¹, 1000 ng mL⁻¹ or 2000 ng mL⁻¹ of FLuc mRNA encapsulated LNPs, GA LNPs, CAT LNPs, EGCG LNPs, TA LNPs, and naked EPO mRNA were added into the seeded B16-F10 and DC 2.4 cells followed by incubation for 24 h. Subsequently, the cell media was aspirated and the concentration of human EPO protein was characterized by Human EPO ELISA kits (Thermo Fisher Scientific) followed by manufacturer's protocol.

### Cell Viability by XTT Assay

B16-F10 and DC 2.4 cells were grown in DMEM or RPMI supplemented with 10% fetal bovine serum (FBS) and 1% penicillin-streptomycin at 37° C. in a 5% CO₂ incubator. The cells were seeded in 96-well Transwell plates at a density of 1×10⁴ cells per well in 100 μL media and incubated overnight. Next day, the cell media was removed and 100 μL of fresh cell media (for cell control), 100 μL of 10× triton dissolved in DMEM or RPMI (as a negative “kill” control to ensure the assay was properly functioning) and 100 μL of LNPs, GA LNPs, CAT LNPs, EGCG LNPs, TA LNPs with 500 ng mL⁻¹ 1000 ng mL⁻¹ or 2000 ng mL⁻¹ of mRNA were introduced into cells for desired time (2, 4, 24 and 48 h). Cells treated with 100 μL of 500 ng mL⁻¹ 1000 ng mL⁻¹ or 2000 ng mL⁻¹ of naked mRNA were used as positive control. Next day, the activated XTT reagent was prepared by mixing 9 mL of 0.2 mg mL⁻¹ XTT in cell media and 22.5 μL of 0.6 mg mL⁻¹ PMS in DPBS). The old media was aspirated and replaced with 100 μL of activated XTT media for 3 h. Finally, the absorbance of the solution was measured using a SpectraMax microplate reader with 475 nm wavelength with a reference wavelength of 675 nm. Untreated cells containing only DMEM/RPMI media were used as background controls. The cell viability results were then calculated by normalizing absorbance to the untreated cells.

### Cell Association

B16-F10 and DC 2.4 cells were seeded in a 24-well transparent well plate at a density 8× 10⁴ cells per well and incubated in complete DMEM/RPMI media at 37° C. for 24 h. Subsequently, 400 μL of 500 ng mL⁻¹ FLuc mRNA encapsulated ATTO-488 labelled LNPs, GA LNPs, CAT LNPs, EGCG LNPs, TA LNPs were added to the cells followed by incubation for desired time (2, 4, 24 and 48 h) at 37° C. After desired times, the cells were washed with DPBS three times and dissociated using trypsin-EDTA. Finally, the samples are analyzed on Attune Flow Cytometer (Thermofisher Scientific, USA). The cell association was assessed by using the percentage of cell with stronger fluorescence intensity than the control untreated cells.

### Intracellular Trafficking Evaluated by Confocal Microscopy

B16-F10 and DC 2.4 cells were seeded at a density of 4×10⁴ cells per well in u-slide 8-well coverslip slides at 37° C. overnight to allow the cellular adhesion to the substrate. After 24 h, the cell media was aspirated, and 300 μL of ATTO-488 labelled LNPs, GA LNPs, CAT LNPs, EGCG LNPs, TA LNPs were added into the cells at a mRNA concentration of 500 ng mL⁻¹, followed by incubation at 37° C. After desired times (2, 4, 24 and 48 h), the supernatant was removed, and cells were washed with DPBS three times. Then 300 μL AF594-WGA (5 μg mL⁻¹) was introduced and incubated for 15 min to stain the cell membrane at 37° C. Subsequently, cells were washed with DPBS for three times followed by 1 μg mL⁻¹ of Hoechst 33342 was added to cells for 10 min to stain the nucleus at 37° C. Finally, the live cell imaging was performed by confocal laser scanning microscope (CLSM) (Leica SP8X, USA) with 63× oil immersion objective. The images were further processed by WCIF Image J software.

### Mechanism of Internalization of LNPs

DC 2.4 cells were seeded in a 24-well transparent well plate at a density 8×10⁴ cells per well and incubated in complete RPMI media at 37° C. for 24 h. Subsequently, Endocytosis inhibitors (pitstop 2, EIPA, filipin from S. filipinensis, NaN3, and cytochalasin D) were added to the cells to achieve final concentrations of 5, 15, 5 μg mL⁻¹, 16.7 mM, and 20 μM, respectively. After 15 min incubation with the endocytosis inhibitors, 300 μL of 500 ng mL⁻¹ FLuc mRNA encapsulated ATTO-488 labelled LNPs, GA LNPs, CAT LNPs, EGCG LNPs, TA LNPs were added to the cells followed by incubation for 2 h at 37° C. Then the cell media was removed, and the cells were washed with DPBS three times and dissociated using trypsin-EDTA. Finally, the cells were analyzed using an Attune Flow Cytometer (Thermofisher Scientific, USA).

### Endosomal Escape of Lipid Nanoparticles

DC 2.4 cells were seeded at a density of 4×10⁴ cells per well in u-slide 8-well coverslip slides at 37° C. overnight to allow the cellular adhesion to the substrate. Next day, the cell media was aspirated and 300 μL of ATTO-488 labelled LNPs, GA LNPs, CAT LNPs, EGCG LNPs, TA LNPs were added into the cells at a FLuc mRNA concentration of 500 ng mL⁻¹, followed by incubation at 37° C. After 24 h, the cells were washed with DPBS three times, and 300 μL 100 nM of LysoTracker Deep Red, dissolved in culture media (prewarmed at 37° C.), was introduced and incubated for 1 h. Subsequently, cells were washed with DPBS three times, and 1 μg mL⁻¹ of Hoechst 33342 was added to cells for 10 min to stain the nucleus. Finally, the live cell imaging was performed by confocal laser scanning microscope (CLSM) (Leica SP8X, USA) with 63× oil immersion objective. The images were further processed by WCIF Image J software. Experiments were performed in triplicates, five representative cell images (>50 cells) were used to calculate the PCC value.

Mechanism Study with Bafilomycin A₁

DC 2.4 cells were seeded at a density of 4×10⁴ cells per well in u-slide 8-well coverslip slides at 37° C. overnight to allow the cellular adhesion to the substrate. Then, the culture media was replaced with 180 μL of fresh media or media containing bafilomycin A₁. Calcein (20 μL, 1.5 mg/mL) was added to each well to obtain a final concentration of 150 μg mL⁻¹ for calcein and 100 nM for bafilomycin A₁. Non-fluorescently labeled LNPs, GA LNPs, CAT LNPs, EGCG LNPs, TA LNPs were incubated with the treated cells for 24 h at a FLuc mRNA concentration of 500 ng mL⁻¹. After incubation, the samples were gently washed three times with DPBS to remove excess materials. Finally, cells were imaged and captured by CLSM with 40× oil immersion objective.

### Buffering Capacity of Lipid Nanoparticles and Complexes

For titration in lipid nanoparticle suspensions, 100 μL of FLuc encapsulated LNPs, GA LNPs, CAT LNPs, EGCG LNPs, TA LNPs were prepared and dispersed in 4.9 mL of Milli-Q water. The pH was subsequently adjusted to ˜7.4 to mimic physiological pH. Then, each LNP formulation was gradually added 5 μL of 0.1 M hydrochloric acid (HCl) dropwise to the nanoparticle suspensions followed by stirring, and the corresponding pH was measured using a Orion Star A214 pH meter (Themo Scientific).

Animal Studies of FLuc mRNA Encapsulated LNPs

All animal studies were approved by the UNC Institutional Animal Care and Use Committee, were consistent with local, state, and federal regulations as applicable, and were supported within the UNC Lineberger ASC at the University of North Carolina at Chapel Hill.

FLuc mRNA encapsulated LNPs, GA LNPs, CAT LNPs, EGCG LNPs, TA LNPs were prepared. Only FLuc mRNA injection was used as a positive control and PBS injection was used as a negative control. Each group was then injected via the tail vein of female C57 black 6 mice (Jackson Laboratory, 18-21 g) at a dose of 0.6 mg kg-1. After 24 hours, mice were injected intraperitoneally with 130 μL of D-luciferin (30 mg mL⁻¹ in PBS). After 15 min, mice were euthanized and organs (pancreas, spleen, liver, kidneys, ovaries, lung and heart) were removed and imaged with an in-vivo imaging system (IVIS) (Perkin Elmer, Waltham, MA).

Luminescence was quantified using AuRA software (Spectral instruments imaging). The liver, spleen, and lung were then fixed in 10% neutral buffered formalin after imaging and were then routinely processed to paraffin. Histological sections were evaluated using hematoxylin and eosin stain. Briefly, Paraffin embedded tissue blocks were sectioned at 5 μm onto positively charged slides. In order to proceed with histological staining, samples were first baked at 60 degrees Celsius for 60 minutes minimum and were then deparaffinized in xylene and hydrated with graded ethanol before continuing with the H&E stain. H&E stains were performed using the autostainer XL from Leica Biosystems. The sections were stained with Hematoxylin (Richard-Allen Scientific, 7211) for 2 mins and Eosin-Y (Richard-Allen Scientific, 7111) for 1 min. Clarifier 2 (7402) and Bluing (7111) solutions from Richard-Allen Scientific were used to differentiate the reaction. After staining, slides were then dehydrated and coverslipped with Cytoseal 60 (8310-4, Thermo Fisher Scientific). Histological analysis was then performed on each sample.

### Animal Blooding Studies

FLuc mRNA encapsulated LNPs, GA LNPs, CAT LNPs, EGCG LNPs, TA LNPs were prepared were prepared. FLuc mRNA injection was used as a positive control and PBS injection was used as a negative control. Each group was then injected via the tail vein of female C57 black 6 mice (Jackson Laboratory, 18-21 g) at a dose of 0.6 mg kg-1. After 24 hours, mice were injected intraperitoneally with 130 μL of D-luciferin (30 mg mL⁻¹ in PBS). 250 μL blood to be collected from each animal by submandibular bleed, in which 60 μL into tubes with K₂EDTA (BD microcontainer) and 190 μL into Minicollect tube (Greiner bio-one) at room temperature. Then the serum was collected by centrifuging the blood at 1300 ref for 10 min. Then the complete blood count and liver and kidney functions from the blood in each mouse was evaluated.

Animal Studies of EPO mRNA Encapsulated LNPs

EPO mRNA encapsulated LNPs, GA LNPs, CAT LNPs, EGCG LNPs, TA LNPs were prepared were prepared were prepared. EPO mRNA injection was used as a positive control and PBS injection was used as a negative control. Each group was then injected via the tail vein of female C57 black 6 mice (Jackson Laboratory, 18-21 g) at a dose of 0.3 mg kg-1. After 24 hours, cardiac puncture to be performed to collect the maximal blood sample for each mouse into Minicollect tubes (Greiner bio-one) at room temperature. Then the serum was collected by centrifuging the blood at 1300 ref for 10 min. Then the dilution of each sample were performed and the concentration of human EPO was characterized by Human EPO ELISA kits (Thermo Fisher Scientific) followed by manufacturer's protocol.

The foregoing examples are illustrative of the present invention, and are not to be construed as limiting thereof. Although the invention has been described in detail with reference to preferred embodiments, variations and modifications exist within the scope and spirit of the invention as described and defined in the following claims.

1. A polyphenol lipid nanoparticle (LNP), comprising about 5-95 mol % ionizable lipid, about 5-30 mol % phospholipid, about 30-60 mol % sterol, about 1-5 mol % PEG lipid, and about 0.5-15 mol % polyphenol.

2. The polyphenol LNP, wherein the polyphenol is a flavonoid or a non-flavonoid.

3. The polyphenol LNP of claim 2, wherein the polyphenol is selected from anthocyanins, dihydrochalcones, dihydroflavonols, flavanols, flavanones, flavonols, flavones, isoflavonoids, phenolic acids, xanthones, stilbenes, lignans, tannins, alkylmethoxyphenols, alkylphenols, curcuminoids, furanocoumarins, hydroxybenzaldehydes, hydroxybenzoketones, hydroxycinnamaldehydes, hydroxycoumarins, hydroxyphenylpropenes, methoxyphenols, naphtoquinones, phenolic terpenes, tyrosols, hydroxyphenolic acids, hydroxyphenylpentanoic acids, or any combination thereof.

4. The polyphenol LNP of claim 3, wherein the polyphenol is a tannin, optionally tannic acid.

5. (canceled)

6. The polyphenol LNP of claim 3, wherein the polyphenol is gallic acid (GA), catechin (CAT), or Epigallocatechin gallate (EGCG).

7. The polyphenol LNP of claim 1, wherein the polyphenol has a molecular weight greater than about 400 g/mol.

8. The polyphenol LNP of claim 1, wherein the ionizable lipid is selected from 9-heptadecanyl 8-{(2-hydroxyethyl)[6-oxo-6-(undecyloxy)hexyl]amino}octanoate (SM-102) and/or [(4-hydroxybutyl) azanediyl]di(hexane-6,1-diyl) bis(2-hexyldecanoate) (ALC-0315).

9. The polyphenol LNP of claim 1, wherein the phospholipid is dioleoylphosphatidylethanolamine (DOPE), dioleoylphosphatidylcholine (DOPC), distearoylphosphatidylcholine (DSPC), or any combination thereof.

10. The polyphenol LNP of claim 1, wherein the sterol is cholesterol and/or phytosterol.

11. The polyphenol LNP of claim 1, wherein the PEG lipid is C14-PEG-2000.

12. The polyphenol LNP of claim 1, comprising about 47 mol % SM-102, about 9 mol % DOPE, about 40 mol % cholesterol, about 2 mol % PEG lipid, and about 2 mol % tannic acid; or
about 44 mol % ALC-0315, about 9 mol % DOPE, about 40 mol % cholesterol, about 2 mol % PEG lipid, and about 5 mol % tannic acid; or
about 46 mol % SM-102, about 9 mol % DOPE, about 39 mol % cholesterol, about 1 mol % PEG lipid, and about 4 mol % polyphenol.

13-15. (canceled)

16. The polyphenol LNP of claim 1, further comprising a cargo, optionally wherein the cargo is a biologically active agent, imaging agent, or therapeutic agent.

17. (canceled)

18. The polyphenol LNP of claim 16, wherein the cargo is a nucleic acid, a protein, a complex of a nucleic acid and a protein, a carbohydrate, a lipid, or a small molecule;
optionally wherein the nucleic acid is an RNA such as an mRNA, antisense oligonucleotide, guide RNA, or siRNA.

19-20. (canceled)

21. The polyphenol LNP of claim 16, wherein the cargo is a gene modulating agent, optionally a gene editing system comprising a CRISPR-Cas system, a zinc finger nuclease, a TALEN, or a meganuclease.

22. (canceled)

23. A method of delivering a cargo to a target cell, comprising contacting the target cell with the polyphenol LNP of claim 16.

24. A method of increasing endosomal escape of a lipid nanoparticle (LNP) delivered to a cell comprising contacting the target cell with the polyphenol LNP of claim 16.

25-27. (canceled)

28. A method of treating or preventing a disease in a subject in need thereof, comprising administering a therapeutically effective amount of the polyphenol LNP of claim 16 to the subject, thereby treating or preventing the disease.

29. A method of providing a vaccine to a subject in need thereof, comprising administering a therapeutically effective amount of the polyphenol LNP of claim 16 to the subject, thereby providing the vaccine to the subject.

30. A method of detecting, diagnosing or monitoring a disease or a disorder in a subject in need thereof, comprising administering an effective amount of the polyphenol LNP of claim 16 to the subject, to thereby detect, diagnose or monitor the disease or disorder.

31. A method of making a cargo-loaded nanoparticle comprising:
adding an organic phase comprising a polyphenol, a sterol, a PEG lipid, an ionizable lipid and a phospholipid to an aqueous phase comprising a cargo to make a mixture.

32-53. (canceled)
